SEQUENCE LISTING (238 sequences)

SEQ ID NO: 1 - DNA, 630 bp, Homo sapiens
atgaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctacaacttccagccctggggttattgtcccagaatctagtgaaaataaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggtgaacctctggataattttgaatcactggataatcaggaatttgattctgaagaagaaactgttgaggattctctagtggaagactcagaaattggcacgtgtgctgaaggaactgtatcttcctctacggatgcaaagccatgtatatga

SEQ ID NO: 2 - protein, 209 aa, Homo sapiens
MNPSMKQKQEEIKENIKNSSVPRRTLKMIQPSASGSLVGRENELSAGLSKRKHRNDHLTSTTSSPGVIVPESSENKNLGGVTQESFDLMIKENPSSQYWKEVAEKRRKALYEALKENEKLHKEIEQKDNEIARLKKENKELAEVAEHVQYMAELIERLNGEPLDNFESLDNQEFDSEEETVEDSLVEDSEIGTCAEGTVSSSTDAKPCI

SEQ ID NO: 3 - DNA, 255 bp, Artificial Sequence
  Description of Artificial Sequence Synthetic peptide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggt

SEQ ID NO: 4 - protein, 85 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAELIERLNG

SEQ ID NO: 5 - DNA, 237 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggt

SEQ ID NO: 6 - protein, 79 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAELIERLNG

SEQ ID NO: 7 - DNA, 210 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagca

SEQ ID NO: 8 - protein, 70 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVA

SEQ ID NO: 9 - DNA, 207 bp, Artificial Sequence
  Description of Artificial Sequence Synthetic peptide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagca

SEQ ID NO: 10 - protein, 69 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVA

SEQ ID NO: 11 - DNA, 204 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagca

SEQ ID NO: 12 - protein, 68 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVA

SEQ ID NO: 13 - DNA, 201 bp, Artificial Sequence
  Description of Artificial Sequence Synthetic peptide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagca

SEQ ID NO: 14 - protein, 67 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVA

SEQ ID NO: 15 - DNA, 198 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagca

SEQ ID NO: 16 - protein, 66 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVA

SEQ ID NO: 17 - DNA, 195 bp, Artificial Sequence
  Description of Artificial Sequence Synthetic peptide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagca

SEQ ID NO: 18 - protein, 65 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVA

SEQ ID NO: 19 - protein, 23 aa, Artificial Sequence
  Description of Artificial Sequence Synthetic peptide
RXXALXEALXEXEXXHXEIEXXD

SEQ ID NO: 20 - DNA, 34 bp, Artificial Sequence
  Description of Artificial Sequence Synthetic primer
ggaattccatatgaaaaatcttggaggagtcacc

SEQ ID NO: 21 - DNA, 34 bp, Artificial Sequence
  Description of Artificial Sequence Synthetic primer
ggaattccatatgacccaggagtcatttgatctt

SEQ ID NO: 22 - DNA, 32 bp, Artificial Sequence
  Description of Artificial Sequence Synthetic primer
cgggatccttatgctacttctgccagttcttt

SEQ ID NO: 23 - DNA, 213 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaa

SEQ ID NO: 24 - protein, 71 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAE

SEQ ID NO: 25 - DNA, 210 bp, Artificial Sequence
  Description of Artificial Sequence Synthetic peptide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaa

SEQ ID NO: 26 - protein, 70 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAE

SEQ ID NO: 27 - DNA, 207 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaa

SEQ ID NO: 28 - protein, 69 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAE

SEQ ID NO: 29 - DNA, 204 bp, Artificial Sequence
  Description of Artificial Sequence Synthetic peptide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaa

SEQ ID NO: 30 - protein, 68 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAE

SEQ ID NO: 31 - DNA, 201 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaa

SEQ ID NO: 32 - protein, 67 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAE

SEQ ID NO: 33 - DNA, 198 bp, Artificial Sequence
  Description of Artificial Sequence Synthetic peptide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaa

SEQ ID NO: 34 - protein, 66 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAE

SEQ ID NO: 35 - DNA, 195 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaa

SEQ ID NO: 36 - protein, 65 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAE

SEQ ID NO: 37 - DNA, 216 bp, Artificial Sequence
  Description of Artificial Sequence Synthetic peptide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacat

SEQ ID NO: 38 - protein, 72 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEH

SEQ ID NO: 39 - DNA, 213 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacat

SEQ ID NO: 40 - protein, 71 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEH

SEQ ID NO: 41 - DNA, 210 bp, Artificial Sequence
  Description of Artificial Sequence Synthetic peptide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacat

SEQ ID NO: 42 - protein, 70 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEH

SEQ ID NO: 43 - DNA, 207 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacat

SEQ ID NO: 44 - protein, 69 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEH

SEQ ID NO: 45 - DNA, 204 bp, Artificial Sequence
  Description of Artificial Sequence Synthetic peptide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacat

SEQ ID NO: 46 - protein, 68 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEH

SEQ ID NO: 47 - DNA, 201 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacat

SEQ ID NO: 48 - protein, 67 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEH

SEQ ID NO: 49 - DNA, 198 bp, Artificial Sequence
  Description of Artificial Sequence Synthetic peptide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacat

SEQ ID NO: 50 - protein, 66 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEH

SEQ ID NO: 51 - DNA, 219 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgta

SEQ ID NO: 52 - protein, 73 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHV

SEQ ID NO: 53 - DNA, 216 bp, Artificial Sequence
  Description of Artificial Sequence Synthetic peptide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgta

SEQ ID NO: 54 - protein, 72 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHV

SEQ ID NO: 55 - DNA, 213 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgta

SEQ ID NO: 56 - protein, 71 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHV

SEQ ID NO: 57 - DNA, 210 bp, Artificial Sequence
  Description of Artificial Sequence Synthetic peptide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgta

SEQ ID NO: 58 - protein, 70 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHV

SEQ ID NO: 59 - DNA, 207 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgta

SEQ ID NO: 60 - protein, 69 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHV

SEQ ID NO: 61 - DNA, 204 bp, Artificial Sequence
  Description of Artificial Sequence Synthetic peptide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgta

SEQ ID NO: 62 - protein, 68 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHV

SEQ ID NO: 63 - DNA, 201 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgta

SEQ ID NO: 64 - protein, 67 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHV

SEQ ID NO: 65 - DNA, 222 bp, Artificial Sequence
  Description of Artificial Sequence Synthetic peptide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacag

SEQ ID NO: 66 - protein, 74 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQ

SEQ ID NO: 67 - DNA, 219 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacag

SEQ ID NO: 68 - protein, 73 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQ

SEQ ID NO: 69 - DNA, 216 bp, Artificial Sequence
  Description of Artificial Sequence Synthetic peptide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacag

SEQ ID NO: 70 - protein, 72 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQ

SEQ ID NO: 71 - DNA, 213 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacag

SEQ ID NO: 72 - protein, 71 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQ

SEQ ID NO: 73 - DNA, 210 bp, Artificial Sequence
  Description of Artificial Sequence Synthetic peptide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacag

SEQ ID NO: 74 - protein, 70 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQ

SEQ ID NO: 75 - DNA, 207 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacag

SEQ ID NO: 76 - protein, 69 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQ

SEQ ID NO: 77 - DNA, 204 bp, Artificial Sequence
  Description of Artificial Sequence Synthetic peptide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacag

SEQ ID NO: 78 - protein, 68 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQ

SEQ ID NO: 79 - DNA, 225 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtat

SEQ ID NO: 80 - protein, 75 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQY

SEQ ID NO: 81 - DNA, 222 bp, Artificial Sequence
  Description of Artificial Sequence Synthetic peptide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtat

SEQ ID NO: 82 - protein, 74 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQY

SEQ ID NO: 83 - DNA, 219 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtat

SEQ ID NO: 84 - protein, 73 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQY

SEQ ID NO: 85 - DNA, 216 bp, Artificial Sequence
  Description of Artificial Sequence Synthetic peptide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtat

SEQ ID NO: 86 - protein, 72 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQY

SEQ ID NO: 87 - DNA, 213 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtat

SEQ ID NO: 88 - protein, 71 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQY

SEQ ID NO: 89 - DNA, 210 bp, Artificial Sequence
  Description of Artificial Sequence Synthetic peptide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtat

SEQ ID NO: 90 - protein, 70 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQY

SEQ ID NO: 91 - DNA, 207 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtat

SEQ ID NO: 92 - protein, 69 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQY

SEQ ID NO: 93 - DNA, 228 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatg

SEQ ID NO: 94 - protein, 76 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYM

SEQ ID NO: 95 - DNA, 225 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatg

SEQ ID NO: 96 - protein, 75 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYM

SEQ ID NO: 97 - DNA, 222 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatg

SEQ ID NO: 98 - protein, 74 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYM

SEQ ID NO: 99 - DNA, 219 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatg

SEQ ID NO: 100 - protein, 73 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYM

SEQ ID NO: 101 - DNA, 216 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatg

SEQ ID NO: 102 - protein, 72 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYM

SEQ ID NO: 103 - DNA, 213 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatg

SEQ ID NO: 104 - protein, 71 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYM

SEQ ID NO: 105 - DNA, 210 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatg

SEQ ID NO: 106 - protein, 70 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYM

SEQ ID NO: 107 - DNA, 231 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggca

SEQ ID NO: 108 - protein, 77 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMA

SEQ ID NO: 109 - DNA, 228 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggca

SEQ ID NO: 110 - protein, 76 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMA

SEQ ID NO: 111 - DNA, 225 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggca

SEQ ID NO: 112 - protein, 75 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMA

SEQ ID NO: 113 - DNA, 222 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggca

SEQ ID NO: 114 - protein, 74 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMA

SEQ ID NO: 115 - DNA, 219 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggca

SEQ ID NO: 116 - protein, 73 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMA

SEQ ID NO: 117 - DNA, 216 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggca

SEQ ID NO: 118 - protein, 72 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMA

SEQ ID NO: 119 - DNA, 213 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggca

SEQ ID NO: 120 - protein, 71 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMA

SEQ ID NO: 121 - DNA, 234 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagag

SEQ ID NO: 122 - protein, 78 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAE

SEQ ID NO: 123 - DNA, 231 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagag

SEQ ID NO: 124 - protein, 77 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAE

SEQ ID NO: 125 - DNA, 228 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagag

SEQ ID NO: 126 - protein, 76 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAE

SEQ ID NO: 127 - DNA, 225 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagag

SEQ ID NO: 128 - protein, 75 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAE

SEQ ID NO: 129 - DNA, 222 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagag

SEQ ID NO: 130 - protein, 74 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAE

SEQ ID NO: 131 - DNA, 219 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagag

SEQ ID NO: 132 - protein, 73 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAE

SEQ ID NO: 133 - DNA, 216 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagag

SEQ ID NO: 134 - protein, 72 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAE

SEQ ID NO: 135 - DNA, 237 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagcta

SEQ ID NO: 136 - protein, 79 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAEL

SEQ ID NO: 137 - DNA, 234 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagcta

SEQ ID NO: 138 - protein, 78 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAEL

SEQ ID NO: 139 - DNA, 231 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagcta

SEQ ID NO: 140 - protein, 77 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAEL

SEQ ID NO: 141 - DNA, 228 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagcta

SEQ ID NO: 142 - protein, 76 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAEL

SEQ ID NO: 143 - DNA, 225 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagcta

SEQ ID NO: 144 - protein, 75 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAEL

SEQ ID NO: 145 - DNA, 222 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagcta

SEQ ID NO: 146 - protein, 74 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAEL

SEQ ID NO: 147 - DNA, 219 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagcta

SEQ ID NO: 148 - protein, 73 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAEL

SEQ ID NO: 149 - DNA, 240 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaata

SEQ ID NO: 150 - protein, 80 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAELI

SEQ ID NO: 151 - DNA, 237 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaata

SEQ ID NO: 152 - protein, 79 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAELI

SEQ ID NO: 153 - DNA, 234 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaata

SEQ ID NO: 154 - protein, 78 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAELI

SEQ ID NO: 155 - DNA, 231 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaata

SEQ ID NO: 156 - protein, 77 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAELI

SEQ ID NO: 157 - DNA, 228 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaata

SEQ ID NO: 158 - protein, 76 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAELI

SEQ ID NO: 159 - DNA, 225 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaata

SEQ ID NO: 160 - protein, 75 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAELI

SEQ ID NO: 161 - DNA, 222 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaata

SEQ ID NO: 162 - protein, 74 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAELI

SEQ ID NO: 163 - DNA, 243 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagag

SEQ ID NO: 164 - protein, 81 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAELIE

SEQ ID NO: 165 - DNA, 240 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagag

SEQ ID NO: 166 - protein, 80 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAELIE

SEQ ID NO: 167 - DNA, 237 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagag

SEQ ID NO: 168 - protein, 79 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAELIE

SEQ ID NO: 169 - DNA, 234 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagag

SEQ ID NO: 170 - protein, 78 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAELIE

SEQ ID NO: 171 - DNA, 231 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagag

SEQ ID NO: 172 - protein, 77 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAELIE

SEQ ID NO: 173 - DNA, 228 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagag

SEQ ID NO: 174 - protein, 76 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAELIE

SEQ ID NO: 175 - DNA, 225 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagag

SEQ ID NO: 176 - protein, 75 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAELIE

SEQ ID NO: 177 - DNA, 246 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagaga

SEQ ID NO: 178 - protein, 82 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAELIER

SEQ ID NO: 179 - DNA, 243 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagaga

SEQ ID NO: 180 - protein, 81 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAELIER

SEQ ID NO: 181 - DNA, 240 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagaga

SEQ ID NO: 182 - protein, 80 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAELIER

SEQ ID NO: 183 - DNA, 237 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagaga

SEQ ID NO: 184 - protein, 79 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAELIER

SEQ ID NO: 185 - DNA, 234 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagaga

SEQ ID NO: 186 - protein, 78 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAELIER

SEQ ID NO: 187 - DNA, 231 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagaga

SEQ ID NO: 188 - protein, 77 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAELIER

SEQ ID NO: 189 - DNA, 228 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagaga

SEQ ID NO: 190 - protein, 76 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAELIER

SEQ ID NO: 191 - DNA, 249 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactg

SEQ ID NO: 192 - protein, 83 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAELIERL

SEQ ID NO: 193 - DNA, 246 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactg

SEQ ID NO: 194 - protein, 82 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAELIERL

SEQ ID NO: 195 - DNA, 243 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactg

SEQ ID NO: 196 - protein, 81 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAELIERL

SEQ ID NO: 197 - DNA, 240 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactg

SEQ ID NO: 198 - protein, 80 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAELIERL

SEQ ID NO: 199 - DNA, 237 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactg

SEQ ID NO: 200 - protein, 79 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAELIERL

SEQ ID NO: 201 - DNA, 234 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactg

SEQ ID NO: 202 - protein, 78 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAELIERL

SEQ ID NO: 203 - DNA, 231 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactg

SEQ ID NO: 204 - protein, 77 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAELIERL

SEQ ID NO: 205 - DNA, 252 bp, Artificial Sequence
  Description of Artificial Sequence Synthetic polynucleotide
aaaaatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaat

SEQ ID NO: 206 - protein, 84 aa, Artificial Sequence
  Description of Artificial Sequence Synthetic peptide
KNLGGVTQESFDLMIKENPSSQYWKEVAEKRRKALYEALKENEKLHKEIEQKDNEIARLKKENKELAEVAEHVQYMAELIERLN

SEQ ID NO: 207 - DNA, 249 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaat

SEQ ID NO: 208 - protein, 83 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAELIERLN

SEQ ID NO: 209 - DNA, 246 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaat

SEQ ID NO: 210 - protein, 82 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAELIERLN

SEQ ID NO: 211 - DNA, 243 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaat

SEQ ID NO: 212 - protein, 81 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAELIERLN

SEQ ID NO: 213 - DNA, 240 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaat

SEQ ID NO: 214 - protein, 80 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAELIERLN

SEQ ID NO: 215 - DNA, 237 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaat

SEQ ID NO: 216 - protein, 79 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAELIERLN

SEQ ID NO: 217 - DNA, 234 bp, Artificial Sequence
  Description of Artificial Sequence Synthetic polynucleotide
acccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaat

SEQ ID NO: 218 - protein, 78 aa, Artificial Sequence
  Description of Artificial Sequence Synthetic peptide
TQESFDLMIKENPSSQYWKEVAEKRRKALYEALKENEKLHKEIEQKDNEIARLKKENKELAEVAEHVQYMAELIERLN

SEQ ID NO: 219 - DNA, 252 bp, Artificial Sequence
  Description of Artificial Sequence Synthetic polynucleotide
aatcttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggt

SEQ ID NO: 220 - protein, 84 aa, Artificial Sequence
  Description of Artificial Sequence Synthetic peptide
NLGGVTQESFDLMIKENPSSQYWKEVAEKRRKALYEALKENEKLHKEIEQKDNEIARLKKENKELAEVAEHVQYMAELIERLNG

SEQ ID NO: 221 - DNA, 249 bp, Artificial Sequence
  Description of Artificial Sequence Synthetic polynucleotide
cttggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggt

SEQ ID NO: 222 - protein, 83 aa, Artificial Sequence
  Description of Artificial Sequence Synthetic peptide
LGGVTQESFDLMIKENPSSQYWKEVAEKRRKALYEALKENEKLHKEIEQKDNEIARLKKENKELAEVAEHVQYMAELIERLNG

SEQ ID NO: 223 - DNA, 246 bp, Artificial Sequence
  Description of Artificial Sequence Synthetic polynucleotide
ggaggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggt

SEQ ID NO: 224 - protein, 82 aa, Artificial Sequence
  Description of Artificial Sequence Synthetic peptide
GGVTQESFDLMIKENPSSQYWKEVAEKRRKALYEALKENEKLHKEIEQKDNEIARLKKENKELAEVAEHVQYMAELIERLNG

SEQ ID NO: 225 - DNA, 243 bp, Artificial Sequence
  Description of Artificial Sequence Synthetic polynucleotide
ggagtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggt

SEQ ID NO: 226 - protein, 81 aa, Artificial Sequence
  Description of Artificial Sequence Synthetic peptide
GVTQESFDLMIKENPSSQYWKEVAEKRRKALYEALKENEKLHKEIEQKDNEIARLKKENKELAEVAEHVQYMAELIERLNG

SEQ ID NO: 227 - DNA, 240 bp, Artificial Sequence
  Description of Artificial Sequence Synthetic polynucleotide
gtcacccaggagtcatttgatcttatgattaaagaaaatccatcctctcagtattggaaggaagtggcagaaaaacggagaaaggcgctgtatgaagcacttaaggaaaatgagaaacttcataaagaaattgaacaaaaggacaatgaaattgcccgcctgaaaaaggagaataaagaactggcagaagtagcagaacatgtacagtatatggcagagctaatagagagactgaatggt

SEQ ID NO: 228 - protein, 80 aa, Artificial Sequence
  Description of Artificial Sequence Synthetic peptide
VTQESFDLMIKENPSSQYWKEVAEKRRKALYEALKENEKLHKEIEQKDNEIARLKKENKELAEVAEHVQYMAELIERLNG

SEQ ID NO: 229 - DNA, 32 bp, Artificial Sequence
  Description of Artificial Sequence Synthetic primer
cgggatccttaaccattcagtctctctattag

SEQ ID NO: 230 - protein, 7 aa, Artificial Sequence
  Description of Artificial Sequence Synthetic peptide
LNIILNA

SEQ ID NO: 231 - protein, 5 aa, Artificial Sequence
  Description of Artificial Sequence Synthetic peptide
ALKLL

SEQ ID NO: 232 - protein, 6 aa, Artificial Sequence
  Description of Artificial Sequence Synthetic 6xHis tag
HHHHHH

SEQ ID NO: 233 - protein, 4 aa, Artificial Sequence
  Description of Artificial Sequence Synthetic peptide
RKKR

SEQ ID NO: 234 - protein, 85 aa, Mus musculus
NKDVGDLTQEAFDLISKENPSSQYWKEVAEQRRKALYEALKENEKLHKEIEQKDSEIARLRKENKDLAEVAEHVQYMAEIERLSN

SEQ ID NO: 235 - protein, 86 aa, Rattus norvegicus
NNSVGDLTQEACDLMTKENPSSQYWKEVAEQRRKALYEALTENEKLHKEIEQKDSEIARLRKENKDLAEVAEHVQYMAEVIERLSN

SEQ ID NO: 236 - protein, 83 aa, Xenopus laevis
NKDCSSEAYDLMVKETPTCLYWKEVAEERRKALYEALQENEKLHKEIELKDEEIARLKQENDELMELAGHVQYMANMIERLTG

SEQ ID NO: 237 - protein, 83 aa, Xenopus laevis
NKDCPSEAYDLMVKETPTCLYWKDVAEERRKALYEALQENEKLHQEIELKDEEIARLKQENDELMELAGHVQYMANMIERLTG

SEQ ID NO: 238 - protein, 87 aa, Danio rerio
NENQPEGVTQEAYELMIKETPGSSYWKEVAEERGKALFSVLQENEKLHKDIEAKDEQIAQLKTENEELQELAQHVQHMADMIERLTG

(19) United States
(12) Patent Application Publication
(10) Pub. No.: US 2008/0279855 A1
(43) Pub. Date: Nov. 13, 2008

(54) Dna Replication Modulating Peptides, Nucleic Acids Encoding Them, and Their Use in Pharmaceutical Compositions

(75) Inventors: Marcel Mechali, Montferrier Sur Lez (FR); Domenico Maiorano, Saint-Martin De Londres (FR); Andre Padilla, Lauret (FR)

Correspondence Address: YOUNG & THOMPSON, 209 Madison Street, Suite 500, ALEXANDRIA, VA, 22314, US

(73) Assignees: CENTRE NATIONAL DE LA RECHERCHE SCIENTIFIQUE, Paris Cedex 16 (FR); UNIVERSITE DE MONTPELLIER, Montpellier Cedex 1 (FR)

(21) Appl. No.: 11/663,445

(22) PCT Filed: Sep. 22, 2004
(86) PCT No.: PCT/EP2004/011438

§ 371 Date: Mar. 12, 2008

(51) Int. Cl. A61K 39/395; A61K 38/00; A61K 31/70; C07K 14/00; C12N 15/11; C12N 15/00; C12N 5/04; C12N 5/06; C12N 1/20; A61P 43/00

(52) U.S. Cl. 424/139.1; 514/12; 514/44; 530/350; 536/23.1; 435/320.1; 435/419; 435/325; 435/252.3

(57) ABSTRACT

The present invention relates to the use of a peptidic sequence which comprises or is constituted of a peptidic chain of at least contiguous amino acids selected from the amino acid sequence SEQ ID NO: 4, SEQ ID NO: 4 being delimited by the amino acid in position 76 and by the amino acid in position 160 of SEQ ID NO: 2 (human Geminin), provided that, if present, the flanking regions of the peptidic chain in the peptidic sequence are different from the flanking regions of the peptidic chain in SEQ ID NO: 2, for the preparation of a drug intended for the treatment or prevention of pathologies implying pathological DNA replication and/or differentiation disorders, or disturbance of the cellular proliferation/differentiation balance.

The present invention relates to peptides modulating DNA replication, nucleic acids encoding them, and their use in pharmaceutical compositions.

Geminin, a polypeptide of about 25 kDa, occurs in the nuclei of higher eukaryotes and functions as both a negative regulator of genome replication and coordinator of differentiation. Geminin was discovered as a protein that is degraded when cells exit from mitosis, by the large ubiquitin-ligase complex known as the cyclosome or anaphase-promoting complex, APC. Geminin tightly interacts with CDT1^{1; 2; 3; 4}, a factor necessary for the recruitment of MCM helicase complex and inhibits the loading of this complex on chromatin. The destruction of Geminin at mitotic exit releases CDT1, which can then serve to reload MCM proteins on chromatin. In Hela cells, Geminin is synthesized throughout the cell cycle, but the protein has a half-life of 3-4 h during the S phase, becomes phosphorylated (at amino-acid residues serine-45 and serine-49, an area closely adjacent to the destruction box motif) as S phase proceeds, and is degraded^{2; 5; 6}. The role of Geminin in embryonic development has also been recently investigated^{7; 8}. These studies demonstrated that murine Geminin associates with members of the Hox-repressing polycomb complex, with the chromatin of Hox regulatory DNA elements and with Hox proteins⁸, Geminin and Six3 transcription factor act antagonistically to control the balance between proliferation and differentiation, and probably Six3 competes with CDT1 binding to Geminin⁷.

The analysis of deletion mutants of Geminin^{6; 9} have defined three almost independent regions of the protein. A destruction box for ubiquitin-mediated degradation during mitosis at the N-terminus, followed by a neuralization domain and at the C-terminus the DNA replication inhibition domain containing a conserved Leucine Zipper (LZ), which corresponds in particular to residues 87 to 168 in Xenopus Geminin⁶ (U.S. Pat. No. 6,548,290). This domain is highly conserved among vertebrates. The corresponding region of human Geminin has a predicted coiled-coil motif of five heptad repeats flanked by an N-terminal sequence rich in basic amino-acids and a C-terminus predicted to form a helix. Coiled-coil structural motifs appear widely distributed in proteins, and genome database searches with coiled-coil prediction programs suggest that 3-5% of all protein residues exist as coiled-coils¹⁰. They are oligomerization motifs commonly occurring at the interface between separate protein chains. They are found in many cytoskeletal and contractile systems (e.g. intermediate filaments, nuclear lamins, and myosins), transcription regulators (e.g. Myc and Max, Fos and Jun, GCN4), viral envelope proteins (e.g. MoMLV, HUV, SIV, influenza)¹¹. Less is known, however, about the structure of Geminin leucine zipper (LZ). The sequence of Geminin-LZs show the predominance of polar residues. This amino-acid distribution has been linked to “natively unfolded” proteins, which lack stable conformational order under physiological conditions¹².

Several peptides derived from human Geminin have been suggested to inhibit DNA replication. However, either their capacity to do so has never been soundly assessed, or their length is too great to contemplate an in vivo use.

Thus, an object of the present invention is to provide new, alternative peptides, liable to bind to Cdt1 and their use to inhibit DNA replication.

Another object of the present invention is to provide nucleic acids encoding for such peptides.

A further object of the present invention is also to provide compounds liable to inhibit the binding of said peptides to Cdt1 and to modulate DNA replication.

The present invention relates to the use of
a peptidic sequence which comprises or is constituted of a peptidic chain of at least 65 contiguous amino acids selected from the amino acid sequence SEQ ID NO: 4, SEQ ID NO: 4 being delimited by the amino acid in position 76 and by the amino acid in position 160 of SEQ ID NO: 2, provided that, if present, the flanking regions of said peptidic chain in said peptidic sequence are different from the flanking regions of said peptidic chain in SEQ ID NO: 2, or
a peptidic sequence derived from the above-defined peptidic sequence by insertion, deletion or substitution of at least one amino acid in said peptidic chain, provided that the resulting derived peptidic chain has a maximum length of 85 amino acids and a minimum length of 65 amino acids, and provided that said peptidic sequence is liable to inhibit DNA replication, and/or to promote cellular differentiation, or
a peptidic sequence presenting a sequence identity of at least 30% with one of the above defined sequences, provided that said peptidic sequence is liable to inhibit DNA replication and/or to provide cellular differentiation, said peptidic sequences being optionally in the form of a dimer, or
an antibody directed to at least one of said peptidic sequences, or
a nucleic acid sequence coding for one at least of said peptidic sequences, or its complementary sequences,
   for the preparation of a drug intended for the treatment or prevention of pathologies implying pathological DNA replication and/or differentiation disorders, or disturbance of the cellular proliferation/differentiation balance.

SEQ ID NO: 2 corresponds to the total amino acid sequence of human Geminin.

As intended herein the expression “the flanking regions of said peptidic chain in said peptidic sequence are different from the flanking regions of said peptidic chain in SEQ ID NO: 2” means that the peptides according to the invention contain at most the sequence of contiguous amino acids extending from amino acid at position 76 to amino acid at position 160 of SEQ ID NO: 2. In particular, the peptides according to the invention do not comprise sequences which comprise the 76-160 sequence of SEQ ID NO: 2 and extend downstream from amino acid 76 and/or upstream from amino acid 160.

The capacity of a compound to inhibit DNA replication can be measured as described in Example 3, by following the general method given in Blow, J. J. & Laskey, R. A. (1986). Initiation of DNA replication in nuclei and purified DNA by a cell-free extract of Xenopus eggs. Cell 47, 577-87

The capacity of a compound to promote cellular differentiation can be measured as described in McBurney M W, Jones-Villeneuve E M, Edwards M K, Anderson P J. 1982. Nature 299, 165-7 Control of muscle and neuronal differentiation in a cultured embryonal carcinoma cell line.

The expression “dimer” relates to the association of two peptides according to the invention together. In particular, said two peptides are associated through non-covalent binding and share the same amino-acid sequence. More particularly, the tridimensional structure of said dimer adopts a coiled coil fold.

As intendended herein the expression “differentiation disorders” relates to developmental abnormalities, such as eye development.

As intendended herein the expression “disturbance of the cellular proliferation/differentiation balance” relates to cancer, organ development such as eye development and apoptosis.

Advantageously, the peptides according to the invention are liable to bind to Cdt1 and, by this way, to prevent the binding of human Geminin to Cdt1, thus impairing the onset of DNA replication.

Similarly, antibodies directed against the peptides according to the invention are liable to block the productive binding of Geminin to Cdt1, by binding to the Cdt1 binding site of Geminin.

Advantageously, the complementary sequence of the above-defined nucleic acid coding for one at least of the peptidic sequences according to the invention are laible to impair the translation of mRNAs encoding geminin.

In a preferred embodiment said peptidic sequence derived from the above-defined peptidic sequence by insertion, deletion or substitution of at least one amino acid in said peptidic chain is such that it presents an identity percentage of at least 30%, in particular at least 50%, more particularly at least 70%, with said peptidic sequence.

According to a preferred embodiment, the invention relates to the above-mentioned use, wherein the peptidic sequence derived from the peptidic sequence defined above by insertion, deletion, or substitution of at least one amino acid in the peptidic chain defined above, is such that the amino acids corresponding or homologous to the amino acids in positions 106, 109, 110, 112, 113, 114, 116, 118, 121, 123, 124, 125, and 128, of SEQ ID NO: 2 are not mutated.

This means that, in a preferred embodiment, the peptides according to the invention comprise the following amino-acid sequence:

| | |
|---|---|
| RX₁X₂ALX₃EALX₄EX₅EX₆X₇HX₈EIEX₉X₁₀D | (SEQ ID NO: 19) |

wherein X₁ to X₁₀ represent any amino-acid.

According to the invention, two amino acids belonging to two different sequences are said to correspond to each other or to be homologous if they can be aligned by using a sequence alignment algorithm such as defined in Altschul et al., Nucleic Acids Res. (1997) 25:3389 or by using the Clustal W software, well known from the man skilled in the art and described in Thompson et al., Nucleic Acids Res. (1994) 22:4673-4680, for instance.

According to another preferred embodiment, the invention relates to the use of a peptidic sequence as defined above, or of a nucleic acid sequence as defined above, for the preparation of a drug intended for the treatment of diseases involving pathological cell proliferation, such as cancers, or for the treatment of diseases involving impaired cell differentiation such as developmental abnormalities.

As intended herein, the expression “developmental abnormalities” relates to incomplete or damaged brain development, and/or eye development

The present invention also relates to the use of an antibody as defined above, or of an antisense of a nucleic acid sequence as defined above, for the preparation of a drug intended for the treatment of diseases involving cellular degeneracy, such as abnormal apopoptosis, Parkinson's disease, Alzheimer disease, multiple sclerosis, spinal cord injury, cellular dedifferentiation, autism, mental retardation or vascular lesion formation.

Such pathologies are well known to the man skilled in the art.

The peptides according to the invention can be administered to an individual at a unit dose ranging from about 1 mg to about 50 mg. As intended herein, the unit dose is defined for an average individual weighting approximately 70 kg.

The present invention also relates to a pharmaceutical composition, comprising as active substance:
a peptidic sequence which comprises or is constituted of a peptidic chain of at least 65 contiguous amino acids selected from the amino acid sequence SEQ ID NO: 4, SEQ ID NO: 4 being delimited by the amino acid in position 76 and by the amino acid in position 160 of SEQ ID NO: 2, provided that, if present, the flanking regions of said peptidic chain in said peptidic sequence are different from the flanking regions of said peptidic chain in SEQ ID NO: 2, or
a peptidic sequence derived from the above-defined peptidic sequence by insertion, deletion or substitution of at least one amino acid in said peptidic chain, provided that the resulting derived peptidic chain has a maximum length of 85 amino acids and a minimum length of 65 amino acids, and provided that said peptidic sequence is liable to inhibit DNA replication, and/or to promote cellular differentiation, the resulting derived peptidic sequence being in particular such that the amino acids corresponding or homologous to the amino acids in positions 106, 109, 110, 112, 113, 114, 116, 118, 121, 123, 124, 125, and 128, of SEQ ID NO: 2 are not mutated, or
a peptidic sequence presenting a sequence identity of at least 30% with one of the above-defined sequences, provided that said peptidic sequence is liable to inhibit DNA replication and/or to provide cellular differentiation, said peptidic sequences being optionally in the form of a dimmer, or
an antibody directed against one of said sequences,
   in association with a pharmaceutically acceptable vehicle.

In a preferred embodiment of the above-defined pharmaceutical composition, the peptidic sequence comprises or is constituted of one of the following amino acid chains:
SEQ ID NO: 4 delimited by amino acid in position 76 and amino acid in position 160 in SEQ ID NO: 2,
SEQ ID NO: 6 delimited by amino acid in position 82 and amino acid in position 160 in SEQ ID NO: 2,
SEQ ID NO: 8 delimited by amino acid in position 76 and amino acid in position 145 in SEQ ID NO: 2,
SEQ ID NO: 10 delimited by amino acid in position 77 and amino acid in position 145 in SEQ ID NO: 2,
SEQ ID NO: 12 delimited by amino acid in position 78 and amino acid in position 145 in SEQ ID NO: 2,
SEQ ID NO: 14 delimited by amino acid in position 79 and amino acid in position 145 in SEQ ID NO:2,
SEQ ID NO: 16 delimited by amino acid in position 80 and amino acid in position 145 in SEQ ID NO: 2,
SEQ ID NO: 18 delimited by amino acid in position 81 and amino acid in position 145 in SEQ ID NO: 2,
SEQ ID NO: 24 delimited by amino acid in position 76 and amino acid in position 146 in SEQ ID NO: 2,
SEQ ID NO: 26 delimited by amino acid in position 77 and amino acid in position 146 in SEQ ID NO: 2,
SEQ ID NO: 28 delimited by amino acid in position 78 and amino acid in position 146 in SEQ ID NO: 2,
SEQ ID NO: 30 delimited by amino acid in position 79 and amino acid in position 146 in SEQ ID NO: 2,
SEQ ID NO: 32 delimited by amino acid in position 80 and amino acid in position 146 in SEQ ID NO: 2,
SEQ ID NO: 34 delimited by amino acid in position 81 and amino acid in position 146 in SEQ ID NO: 2,
SEQ ID NO: 36 delimited by amino acid in position 82 and amino acid in position 146 in SEQ ID NO: 2,
SEQ ID NO: 38 delimited by amino acid in position 76 and amino acid in position 147 in SEQ ID NO: 2,
SEQ ID NO: 40 delimited by amino acid in position 77 and amino acid in position 147 in SEQ ID NO: 2,
SEQ ID NO: 42 delimited by amino acid in position 78 and amino acid in position 147 in SEQ ID NO: 2,
SEQ ID NO: 44 delimited by amino acid in position 79 and amino acid in position 147 in SEQ ID NO: 2,
SEQ ID NO: 46 delimited by amino acid in position 80 and amino acid in position 147 in SEQ ID NO: 2,
SEQ ID NO: 48 delimited by amino acid in position 81 and amino acid in position 147 in SEQ ID NO: 2,
SEQ ID NO: 50 delimited by amino acid in position 82 and amino acid in position 147 in SEQ ID NO: 2,
SEQ ID NO: 52 delimited by amino acid in position 76 and amino acid in position 148 in SEQ ID NO: 2,
SEQ ID NO: 54 delimited by amino acid in position 77 and amino acid in position 148 in SEQ ID NO: 2,
SEQ ID NO: 56 delimited by amino acid in position 78 and amino acid in position 148 in SEQ ID NO: 2,
SEQ ID NO: 58 delimited by amino acid in position 79 and amino acid in position 148 in SEQ ID NO: 2,
SEQ ID NO: 60 delimited by amino acid in position 80 and amino acid in position 148 in SEQ ID NO: 2,
SEQ ID NO: 62 delimited by amino acid in position 81 and amino acid in position 148 in SEQ ID NO: 2,
SEQ ID NO: 64 delimited by amino acid in position 82 and amino acid in position 148 in SEQ ID NO: 2,
SEQ ID NO: 66 delimited by amino acid in position 76 and amino acid in position 149 in SEQ ID NO: 2,
SEQ ID NO: 68 delimited by amino acid in position 77 and amino acid in position 149 in SEQ ID NO: 2,
SEQ ID NO: 70 delimited by amino acid in position 78 and amino acid in position 149 in SEQ ID NO: 2,
SEQ ID NO: 72 delimited by amino acid in position 79 and amino acid in position 149 in SEQ ID NO: 2,
SEQ ID NO: 74 delimited by amino acid in position 80 and amino acid in position 149 in SEQ ID NO: 2,
SEQ ID NO: 76 delimited by amino acid in position 81 and amino acid in position 149 in SEQ ID NO: 2,
SEQ ID NO: 78 delimited by amino acid in position 82 and amino acid in position 149 in SEQ ID NO: 2,
SEQ ID NO: 80 delimited by amino acid in position 76 and amino acid in position 150 in SEQ ID NO: 2,
SEQ ID NO: 82 delimited by amino acid in position 77 and amino acid in position 150 in SEQ ID NO:2,
SEQ ID NO: 84 delimited by amino acid in position 78 and amino acid in position 150 in SEQ ID NO: 2,
SEQ ID NO: 86 delimited by amino acid in position 79 and amino acid in position 150 in SEQ ID NO: 2,
SEQ ID NO: 88 delimited by amino acid in position 80 and amino acid in position 150 in SEQ ID NO: 2,
SEQ ID NO: 90 delimited by amino acid in position 81 and amino acid in position 150 in SEQ ID NO: 2,
SEQ ID NO: 92 delimited by amino acid in position 82 and amino acid in position 150 in SEQ ID NO: 2,
SEQ ID NO: 94 delimited by amino acid in position 76 and amino acid in position 151 in SEQ ID NO: 2,
SEQ ID NO: 96 delimited by amino acid in position 77 and amino acid in position 151 in SEQ ID NO: 2,
SEQ ID NO: 98 delimited by amino acid in position 78 and amino acid in position 151 in SEQ ID NO: 2,
SEQ ID NO: 100 delimited by amino acid in position 79 and amino acid in position 151 in SEQ ID NO: 2,
SEQ ID NO: 102 delimited by amino acid in position 80 and amino acid in position 151 in SEQ ID NO: 2,
SEQ ID NO: 104 delimited by amino acid in position 81 and amino acid in position 151 in SEQ ID NO: 2,
SEQ ID NO: 106 delimited by amino acid in position 82 and amino acid in position 151 in SEQ ID NO: 2,
SEQ ID NO: 108 delimited by amino acid in position 76 and amino acid in position 152 in SEQ ID NO: 2,
SEQ ID NO: 110 delimited by amino acid in position 77 and amino acid in position 152 in SEQ ID NO: 2,
SEQ ID NO: 112 delimited by amino acid in position 78 and amino acid in position 152 in SEQ ID NO: 2,
SEQ ID NO: 114 delimited by amino acid in position 79 and amino acid in position 152 in SEQ ID NO: 2,
SEQ ID NO: 116 delimited by amino acid in position 80 and amino acid in position 152 in SEQ ID NO: 2,
SEQ ID NO: 118 delimited by amino acid in position 81 and amino acid in position 152 in SEQ ID NO: 2,
SEQ ID NO: 120 delimited by amino acid in position 82 and amino acid in position 152 in SEQ ID NO: 2,
SEQ ID NO: 122 delimited by amino acid in position 76 and amino acid in position 153 in SEQ ID NO: 2,
SEQ ID NO: 124 delimited by amino acid in position 77 and amino acid in position 153 in SEQ ID NO: 2,
SEQ ID NO: 126 delimited by amino acid in position 78 and amino acid in position 153 in SEQ ID NO: 2,
SEQ ID NO: 128 delimited by amino acid in position 79 and amino acid in position 153 in SEQ ID NO: 2,
SEQ ID NO: 130 delimited by amino acid in position 80 and amino acid in position 153 in SEQ ID NO: 2,
SEQ ID NO: 132 delimited by amino acid in position 81 and amino acid in position 153 in SEQ ID NO:2,
SEQ ID NO: 134 delimited by amino acid in position 82 and amino acid in position 153 in SEQ ID NO: 2,
SEQ ID NO: 136 delimited by amino acid in position 76 and amino acid in position 154 in SEQ ID NO: 2,
SEQ ID NO: 138 delimited by amino acid in position 77 and amino acid in position 154 in SEQ ID NO: 2,
SEQ ID NO: 140 delimited by amino acid in position 78 and amino acid in position 154 in SEQ ID NO: 2,
SEQ ID NO: 142 delimited by amino acid in position 79 and amino acid in position 154 in SEQ ID NO: 2,
SEQ ID NO: 144 delimited by amino acid in position 80 and amino acid in position 154 in SEQ ID NO: 2,
SEQ ID NO: 146 delimited by amino acid in position 81 and amino acid in position 154 in SEQ ID NO: 2,
SEQ ID NO: 148 delimited by amino acid in position 82 and amino acid in position 154 in SEQ ID NO: 2,
SEQ ID NO: 150 delimited by amino acid in position 76 and amino acid in position 155 in SEQ ID NO: 2,
SEQ ID NO: 152 delimited by amino acid in position 77 and amino acid in position 155 in SEQ ID NO: 2,
SEQ ID NO: 154 delimited by amino acid in position 78 and amino acid in position 155 in SEQ ID NO: 2,
SEQ ID NO: 156 delimited by amino acid in position 79 and amino acid in position 155 in SEQ ID NO:2,
SEQ ID NO: 158 delimited by amino acid in position 80 and amino acid in position 155 in SEQ ID NO: 2,
SEQ ID NO: 160 delimited by amino acid in position 81 and amino acid in position 155 in SEQ ID NO: 2,
SEQ ID NO: 162 delimited by amino acid in position 82 and amino acid in position 155 in SEQ ID NO: 2,
SEQ ID NO: 164 delimited by amino acid in position 76 and amino acid in position 156 in SEQ ID NO: 2,
SEQ ID NO: 166 delimited by amino acid in position 77 and amino acid in position 156 in SEQ ID NO: 2,
SEQ ID NO: 168 delimited by amino acid in position 78 and amino acid in position 156 in SEQ ID NO: 2,
SEQ ID NO: 170 delimited by amino acid in position 79 and amino acid in position 156 in SEQ ID NO: 2,
SEQ ID NO: 172 delimited by amino acid in position 80 and amino acid in position 156 in SEQ ID NO: 2,
SEQ ID NO: 174 delimited by amino acid in position 81 and amino acid in position 156 in SEQ ID NO: 2,
SEQ ID NO: 176 delimited by amino acid in position 82 and amino acid in position 156 in SEQ ID NO: 2,
SEQ ID NO: 178 delimited by amino acid in position 76 and amino acid in position 157 in SEQ ID NO: 2,
SEQ ID NO: 180 delimited by amino acid in position 77 and amino acid in position 157 in SEQ ID NO: 2,
SEQ ID NO: 182 delimited by amino acid in position 78 and amino acid in position 157 in SEQ ID NO:2,
SEQ ID NO: 184 delimited by amino acid in position 79 and amino acid in position 157 in SEQ ID NO: 2,
SEQ ID NO: 186 delimited by amino acid in position 80 and amino acid in position 157 in SEQ ID NO: 2,
SEQ ID NO: 188 delimited by amino acid in position 81 and amino acid in position 157 in SEQ ID NO:2,
SEQ ID NO: 190 delimited by amino acid in position 82 and amino acid in position 157 in SEQ ID NO:2,
SEQ ID NO: 192 delimited by amino acid in position 76 and amino acid in position 158 in SEQ ID NO: 2,
SEQ ID NO: 194 delimited by amino acid in position 77 and amino acid in position 158 in SEQ ID NO: 2,
SEQ ID NO: 196 delimited by amino acid in position 78 and amino acid in position 158 in SEQ ID NO:2,
SEQ ID NO: 198 delimited by amino acid in position 79 and amino acid in position 158 in SEQ ID NO: 2,
SEQ ID NO: 200 delimited by amino acid in position 80 and amino acid in position 158 in SEQ ID NO:2,
SEQ ID NO: 202 delimited by amino acid in position 81 and amino acid in position 158 in SEQ ID NO: 2,
SEQ ID NO: 204 delimited by amino acid in position 82 and amino acid in position 158 in SEQ ID NO: 2,
SEQ ID NO: 206 delimited by amino acid in position 76 and amino acid in position 159 in SEQ ID NO: 2,
SEQ ID NO: 208 delimited by amino acid in position 77 and amino acid in position 159 in SEQ ID NO: 2,
SEQ ID NO: 210 delimited by amino acid in position 78 and amino acid in position 159 in SEQ ID NO: 2,
SEQ ID NO: 212 delimited by amino acid in position 79 and amino acid in position 159 in SEQ ID NO: 2,
SEQ ID NO: 214 delimited by amino acid in position 80 and amino acid in position 159 in SEQ ID NO: 2,
SEQ ID NO: 216 delimited by amino acid in position 81 and amino acid in position 159 in SEQ ID NO: 2,
SEQ ID NO: 218 delimited by amino acid in position 82 and amino acid in position 159 in SEQ ID NO: 2,
SEQ ID NO: 220 delimited by amino acid in position 77 and amino acid in position 160 in SEQ ID NO: 2,
SEQ ID NO: 222 delimited by amino acid in position 78 and amino acid in position 160 in SEQ ID NO: 2,
SEQ ID NO: 224 delimited by amino acid in position 79 and amino acid in position 160 in SEQ ID NO: 2,
SEQ ID NO: 226 delimited by amino acid in position 80 and amino acid in position 160 in SEQ ID NO: 2,
SEQ ID NO: 228 delimited by amino acid in position 81 and amino acid in position 160 in SEQ ID NO: 2,
provided that, if present, the flanking regions of said sequences are different from the flanking regions of said sequences in SEQ ID NO: 2, or
a peptidic sequence derived from the above-defined peptidic sequence by insertion, deletion or mutation, of at least one amino acid in said peptidic chains, provided that the resulting derived sequence has a maximum length of 85 amino acids and a minimum length of 65 amino acids, provided that said peptidic sequence is liable to inhibit DNA replication, and/or to promote cellular differentiation, or
a peptidic sequence presenting a sequence identity of at least 30% with one of the above defined peptidic sequences, provided said peptidic sequence is liable to inhibit DNA replication and/or to provide cellular differentiation,
said peptidic sequences being optionally in the form of a dimer,
   in association with a pharmaceutically acceptable vehicle.

In a particularly preferred embodiment of the invention, the above-mentioned pharmaceutical compositions are suitable for the administration of the peptides according to the invention to an individual at a unit dose ranging from about 1 mg to about 50 mg.

As intended herein the unit dose is defined for an average individual weighting approximately 70 kg.

The present invention also relates to a pharmaceutical composition containing, as active substance, a nucleic acid coding for one of the above-defined peptidic sequences, or its complementary sequence, or an antisense of the above-defined nucleic acid, in association with a pharmaceutically acceptable vehicle.

The present invention also relates to a pharmaceutical composition containing as active substance:
a nucleic acid sequence which comprises or is constituted of a nucleotide chain of at least 195 contiguous nucleotides selected from the nucleotide SEQ ID NO: 3, SEQ ID NO: 3 being delimited by the nucleotide in position 226 and by the nucleotide in position 480 of SEQ ID NO: 1, provided that, if present, the flanking regions of said nucleotide chain in said nucleic acid sequence are different from the flanking regions of said nucleotide chain in SEQ ID NO: 1, or
a nucleic acid sequence derived from the above-defined sequence by insertion, deletion or mutation, of at least one nucleotide in said nucleotide chain, provided that the resulting derived nucleic acid sequence has a maximum length of 255 nucleotides and a minimum length of 195 nucleotides, and provided that said derived nucleic acid codes for a peptidic sequence liable to inhibit DNA replication and/or to promote cellular differentiation, the resulting derived nucleic acid sequence being in particular such that it codes for a peptidic sequence in which the amino acids corresponding or homologous to the amino acids in positions 106, 109, 110, 112, 113, 114, 116, 118, 121, 123, 124, 125, and 128, of SEQ ID NO: 2 are not mutated, or
a nucleic acid presenting a sequence identity of at least 33% with one of the above defined nucleic acid sequences, provided that said nucleic acid sequence codes for a peptidic sequence liable to inhibit DNA replication and/or to promote cellular differentiation or its complementary sequence,
or the complementary sequence of one of the above-defined nucleic sequences
or an antisense of the above-defined sequences,
   in association with a pharmaceutically acceptable vehicle.

SEQ ID NO: 1 corresponds to the coding sequence of the human Geminin gene.

In a preferred embodiment of the invention, the above-defined pharmaceutical composition contains, as active substance, a nucleic acid which comprises or is constituted of at least one of the following nucleotide chains:
SEQ ID NO: 3 delimited by the nucleotide in position 226 and the nucleotide in position 480 in SEQ ID NO: 1,
SEQ ID NO: 5 delimited by the nucleotide in position 244 and the nucleotide in position 480 in SEQ ID NO: 1,
SEQ ID NO: 7 delimited by the nucleotide in position 226 and the nucleotide in position 435 in SEQ ID NO: 1,
SEQ ID NO: 9 delimited by the nucleotide in position 229 and the nucleotide in position 435 in SEQ ID NO: 1,
SEQ ID NO: 11 delimited by the nucleotide in position 232 and the nucleotide in position 435 in SEQ ID NO: 1,
SEQ ID NO: 13 delimited by the nucleotide in position 235 and the nucleotide in position 435 in SEQ ID NO: 1,
SEQ ID NO: 15 delimited by the nucleotide in position 238 and the nucleotide in position 435 in SEQ ID NO: 1,
SEQ ID NO: 17 delimited by the nucleotide in position 241 and the nucleotide in position 435 in SEQ ID NO: 1,
SEQ ID NO: 23 delimited by the nucleotide in position 226 and the nucleotide in position 438 in SEQ ID NO: 1,
SEQ ID NO: 25 delimited by the nucleotide in position 229 and the nucleotide in position 438 in SEQ ID NO: 1,
SEQ ID NO: 27 delimited by the nucleotide in position 232 and the nucleotide in position 438 in SEQ ID NO: 1,
SEQ ID NO: 29 delimited by the nucleotide in position 235 and the nucleotide in position 438 in SEQ ID NO: 1,
SEQ ID NO: 31 delimited by the nucleotide in position 238 and the nucleotide in position 438 in SEQ ID NO: 1,
SEQ ID NO: 33 delimited by the nucleotide in position 241 and the nucleotide in position 438 in SEQ ID NO: 1,
SEQ ID NO: 35 delimited by the nucleotide in position 244 and the nucleotide in position 438 in SEQ ID NO: 1,
SEQ ID NO: 37 delimited by the nucleotide in position 226 and the nucleotide in position 441 in SEQ ID NO: 1,
SEQ ID NO: 39 delimited by the nucleotide in position 229 and the nucleotide in position 441 in SEQ ID NO: 1,
SEQ ID NO: 41 delimited by the nucleotide in position 232 and the nucleotide in position 441 in SEQ ID NO: 1,
SEQ ID NO: 43 delimited by the nucleotide in position 235 and the nucleotide in position 441 in SEQ ID NO: 1,
SEQ ID NO: 45 delimited by the nucleotide in position 238 and the nucleotide in position 441 in SEQ ID NO: 1,
SEQ ID NO: 47 delimited by the nucleotide in position 241 and the nucleotide in position 441 in SEQ ID NO: 1,
SEQ ID NO: 49 delimited by the nucleotide in position 244 and the nucleotide in position 441 in SEQ ID NO: 1,
SEQ ID NO: 51 delimited by the nucleotide in position 226 and the nucleotide in position 444 in SEQ ID NO: 1,
SEQ ID NO: 53 delimited by the nucleotide in position 229 and the nucleotide in position 444 in SEQ ID NO: 1,
SEQ ID NO: 55 delimited by the nucleotide in position 232 and the nucleotide in position 444 in SEQ ID NO: 1,
SEQ ID NO: 57 delimited by the nucleotide in position 235 and the nucleotide in position 444 in SEQ ID NO: 1,
SEQ ID NO: 59 delimited by the nucleotide in position 238 and the nucleotide in position 444 in SEQ ID NO: 1,
SEQ ID NO: 61 delimited by the nucleotide in position 241 and the nucleotide in position 444 in SEQ ID NO: 1,
SEQ ID NO: 63 delimited by the nucleotide in position 244 and the nucleotide in position 444 in SEQ ID NO: 1,
SEQ ID NO: 65 delimited by the nucleotide in position 226 and the nucleotide in position 447 in SEQ ID NO: 1,
SEQ ID NO: 67 delimited by the nucleotide in position 229 and the nucleotide in position 447 in SEQ ID NO: 1,
SEQ ID NO: 69 delimited by the nucleotide in position 232 and the nucleotide in position 447 in SEQ ID NO: 1,
SEQ ID NO: 71 delimited by the nucleotide in position 235 and the nucleotide in position 447 in SEQ ID NO: 1, —SEQ ID NO: 73 delimited by the nucleotide in position 238 and the nucleotide in position 447 in SEQ ID NO: 1,
SEQ ID NO: 75 delimited by the nucleotide in position 241 and the nucleotide in position 447 in SEQ ID NO: 1,
SEQ ID NO: 77 delimited by the nucleotide in position 244 and the nucleotide in position 447 in SEQ ID NO: 1,
SEQ ID NO: 79 delimited by the nucleotide in position 226 and the nucleotide in position 450 in SEQ ID NO: 1,
SEQ ID NO: 81 delimited by the nucleotide in position 229 and the nucleotide in position 450 in SEQ ID NO: 1,
SEQ ID NO: 83 delimited by the nucleotide in position 232 and the nucleotide in position 450 in SEQ ID NO: 1,
SEQ ID NO: 85 delimited by the nucleotide in position 235 and the nucleotide in position 450 in SEQ ID NO: 1,
SEQ ID NO: 87 delimited by the nucleotide in position 238 and the nucleotide in position 450 in SEQ ID NO: 1,
SEQ ID NO: 89 delimited by the nucleotide in position 241 and the nucleotide in position 450 in SEQ ID NO: 1,
SEQ ID NO: 91 delimited by the nucleotide in position 244 and the nucleotide in position 450 in SEQ ID NO: 1,
SEQ ID NO: 93 delimited by the nucleotide in position 226 and the nucleotide in position 453 in SEQ ID NO: 1,
SEQ ID NO: 95 delimited by the nucleotide in position 229 and the nucleotide in position 453 in SEQ ID NO: 1,
SEQ ID NO: 97 delimited by the nucleotide in position 232 and the nucleotide in position 453 in SEQ ID NO: 1,
SEQ ID NO: 99 delimited by the nucleotide in position 235 and the nucleotide in position 453 in SEQ ID NO: 1,
SEQ ID NO: 101 delimited by the nucleotide in position 238 and the nucleotide in position 453 in SEQ ID NO: 1,
SEQ ID NO: 103 delimited by the nucleotide in position 241 and the nucleotide in position 453 in SEQ ID NO: 1,
SEQ ID NO: 105 delimited by the nucleotide in position 244 and the nucleotide in position 453 in SEQ ID NO: 1,
SEQ ID NO: 107 delimited by the nucleotide in position 226 and the nucleotide in position 456 in SEQ ID NO: 1,
SEQ ID NO: 109 delimited by the nucleotide in position 229 and the nucleotide in position 456 in SEQ ID NO: 1,
SEQ ID NO: 111 delimited by the nucleotide in position 232 and the nucleotide in position 456 in SEQ ID NO: 1,
SEQ ID NO: 113 delimited by the nucleotide in position 235 and the nucleotide in position 456 in SEQ ID NO: 1,
SEQ ID NO: 115 delimited by the nucleotide in position 238 and the nucleotide in position 456 in SEQ ID NO: 1,
SEQ ID NO: 117 delimited by the nucleotide in position 241 and the nucleotide in position 456 in SEQ ID NO: 1,
SEQ ID NO: 119 delimited by the nucleotide in position 244 and the nucleotide in position 456 in SEQ ID NO: 1,
SEQ ID NO: 121 delimited by the nucleotide in position 226 and the nucleotide in position 459 in SEQ ID NO: 1,
SEQ ID NO: 123 delimited by the nucleotide in position 229 and the nucleotide in position 459 in SEQ ID NO: 1,
SEQ ID NO: 125 delimited by the nucleotide in position 232 and the nucleotide in position 459 in SEQ ID NO: 1,
SEQ ID NO: 127 delimited by the nucleotide in position 235 and the nucleotide in position 459 in SEQ ID NO: 1,
SEQ ID NO: 129 delimited by the nucleotide in position 238 and the nucleotide in position 459 in SEQ ID NO: 1,
SEQ ID NO: 131 delimited by the nucleotide in position 241 and the nucleotide in position 459 in SEQ ID NO: 1,
SEQ ID NO: 133 delimited by the nucleotide in position 244 and the nucleotide in position 459 in SEQ ID NO: 1,
SEQ ID NO: 135 delimited by the nucleotide in position 226 and the nucleotide in position 462 in SEQ ID NO: 1,
SEQ ID NO: 137 delimited by the nucleotide in position 229 and the nucleotide in position 462 in SEQ ID NO: 1,
SEQ ID NO: 139 delimited by the nucleotide in position 232 and the nucleotide in position 462 in SEQ ID NO: 1,
SEQ ID NO: 141 delimited by the nucleotide in position 235 and the nucleotide in position 462 in SEQ ID NO: 1,
SEQ ID NO: 143 delimited by the nucleotide in position 238 and the nucleotide in position 462 in SEQ ID NO: 1,
SEQ ID NO: 145 delimited by the nucleotide in position 241 and the nucleotide in position 462 in SEQ ID NO: 1,
SEQ ID NO: 147 delimited by the nucleotide in position 244 and the nucleotide in position 462 in SEQ ID NO: 1,
SEQ ID NO: 149 delimited by the nucleotide in position 226 and the nucleotide in position 465 in SEQ ID NO: 1,
SEQ ID NO: 151 delimited by the nucleotide in position 229 and the nucleotide in position 465 in SEQ ID NO: 1,
SEQ ID NO: 153 delimited by the nucleotide in position 232 and the nucleotide in position 465 in SEQ ID NO: 1,
SEQ ID NO: 155 delimited by the nucleotide in position 235 and the nucleotide in position 465 in SEQ ID NO: 1,
SEQ ID NO: 157 delimited by the nucleotide in position 238 and the nucleotide in position 465 in SEQ ID NO: 1,
SEQ ID NO: 159 delimited by the nucleotide in position 241 and the nucleotide in position 465 in SEQ ID NO: 1,
SEQ ID NO: 161 delimited by the nucleotide in position 244 and the nucleotide in position 465 in SEQ ID NO: 1,
SEQ ID NO: 163 delimited by the nucleotide in position 226 and the nucleotide in position 468 in SEQ ID NO: 1,
SEQ ID NO: 165 delimited by the nucleotide in position 229 and the nucleotide in position 468 in SEQ ID NO: 1,
SEQ ID NO: 167 delimited by the nucleotide in position 232 and the nucleotide in position 468 in SEQ ID NO: 1,
SEQ ID NO: 169 delimited by the nucleotide in position 235 and the nucleotide in position 468 in SEQ ID NO: 1,
SEQ ID NO: 171 delimited by the nucleotide in position 238 and the nucleotide in position 468 in SEQ ID NO: 1,
SEQ ID NO: 173 delimited by the nucleotide in position 241 and the nucleotide in position 468 in SEQ ID NO: 1,
SEQ ID NO: 175 delimited by the nucleotide in position 244 and the nucleotide in position 468 in SEQ ID NO: 1,
SEQ ID NO: 177 delimited by the nucleotide in position 226 and the nucleotide in position 471 in SEQ ID NO: 1,
SEQ ID NO: 179 delimited by the nucleotide in position 229 and the nucleotide in position 471 in SEQ ID NO: 1,
SEQ ID NO: 181 delimited by the nucleotide in position 232 and the nucleotide in position 471 in SEQ ID NO: 1,
SEQ ID NO: 183 delimited by the nucleotide in position 235 and the nucleotide in position 471 in SEQ ID NO: 1,
SEQ ID NO: 185 delimited by the nucleotide in position 238 and the nucleotide in position 471 in SEQ ID NO: 1,
SEQ ID NO: 187 delimited by the nucleotide in position 241 and the nucleotide in position 471 in SEQ ID NO: 1,
SEQ ID NO: 189 delimited by the nucleotide in position 244 and the nucleotide in position 471 in SEQ ID NO: 1,
SEQ ID NO: 191 delimited by the nucleotide in position 226 and the nucleotide in position 474 in SEQ ID NO: 1, —SEQ ID NO: 193 delimited by the nucleotide in position 229 and the nucleotide in position 474 in SEQ ID NO: 1,
SEQ ID NO: 195 delimited by the nucleotide in position 232 and the nucleotide in position 474 in SEQ ID NO: 1,
SEQ ID NO: 197 delimited by the nucleotide in position 235 and the nucleotide in position 474 in SEQ ID NO: 1,
SEQ ID NO: 199 delimited by the nucleotide in position 238 and the nucleotide in position 474 in SEQ ID NO: 1,
SEQ ID NO: 201 delimited by the nucleotide in position 241 and the nucleotide in position 474 in SEQ ID NO: 1,
SEQ ID NO: 203 delimited by the nucleotide in position 244 and the nucleotide in position 474 in SEQ ID NO: 1,
SEQ ID NO: 205 delimited by the nucleotide in position 226 and the nucleotide in position 477 in SEQ ID NO: 1,
SEQ ID NO: 207 delimited by the nucleotide in position 229 and the nucleotide in position 477 in SEQ ID NO: 1,
SEQ ID NO: 209 delimited by the nucleotide in position 232 and the nucleotide in position 477 in SEQ ID NO: 1,
SEQ ID NO: 211 delimited by the nucleotide in position 235 and the nucleotide in position 477 in SEQ ID NO: 1,
SEQ ID NO: 213 delimited by the nucleotide in position 238 and the nucleotide in position 477 in SEQ ID NO: 1,
SEQ ID NO: 215 delimited by the nucleotide in position 241 and the nucleotide in position 477 in SEQ ID NO: 1,
SEQ ID NO: 217 delimited by the nucleotide in position 244 and the nucleotide in position 477 in SEQ ID NO: 1,
SEQ ID NO: 219 delimited by the nucleotide in position 229 and the nucleotide in position 480 in SEQ ID NO: 1,
SEQ ID NO: 221 delimited by the nucleotide in position 232 and the nucleotide in position 480 in SEQ ID NO: 1,
SEQ ID NO: 223 delimited by the nucleotide in position 235 and the nucleotide in position 480 in SEQ ID NO: 1,
SEQ ID NO: 225 delimited by the nucleotide in position 238 and the nucleotide in position 480 in SEQ ID NO: 1,
SEQ ID NO: 227 delimited by the nucleotide in position 241 and the nucleotide in position 480 in SEQ ID NO: 1,
provided that, if present, the flanking regions of said nucleotide chains in said nucleic acid are different from the flanking regions of said nucleotide chains in SEQ ID NO: 1, or
a nucleic acid sequence derived from the above-defined sequence by insertion, deletion or mutation of at least one nucleotide in said nucleotide chains, provided that the resulting derived nucleic acid sequence has a maximum length of 255 nucleotides and a minimum length of 195 nucleotides, and provided that said derived nucleic acid codes for a peptidic sequence liable to inhibit DNA replication and/or to promote cellular differentiation, the resulting derived peptidic sequence being in particular such that the amino acids corresponding or homologous to the amino acids in positions 106, 109, 110, 112, 113, 114, 116, 118, 121, 123, 124, 125, and 128, of SEQ ID NO: 2 are not mutated, or
a nucleic acid presenting a sequence identity of at least 33% with one of the above-defined sequences, provided that said nucleic acid sequence codes for a peptidic sequence liable to inhibit DNA replication and/or to promote cellular differentiation or its complementary sequence,
or the complementary sequence of one of the above-defined nucleic sequences,
or an antisense of the above-defined nucleic sequences,
   in association with a pharmaceutically acceptable vehicle.

As will be apparent for the man skilled in the art, SEQ ID NO: 2n+1, wherein n is an integer from 0 to 8 and, SEQ ID NO: 2k+1 wherein k is an integer from 11 to 113, respectively encode SEQ ID NO: 2n+2 and SEQ ID NO: 2k+2.

The present invention also relates to a peptide comprising or being constituted by one of the following peptidic chains:
SEQ ID NO: 4 delimited by amino acid in position 76 and amino acid in position 160 in SEQ ID NO: 2,
SEQ ID NO: 6 delimited by amino acid in position 82 and amino acid in position 160 in SEQ ID NO: 2,
SEQ ID NO: 8 delimited by amino acid in position 76 and amino acid in position 145 in SEQ ID NO: 2,
SEQ ID NO: 10 delimited by amino acid in position 77 and amino acid in position 145 in SEQ ID NO: 2,
SEQ ID NO: 12 delimited by amino acid in position 78 and amino acid in position 145 in SEQ ID NO: 2,
SEQ ID NO: 14 delimited by amino acid in position 79 and amino acid in position 145 in SEQ ID NO: 2,
SEQ ID NO: 16 delimited by amino acid in position 80 and amino acid in position 145 in SEQ ID NO: 2,
SEQ ID NO: 18 delimited by amino acid in position 81 and amino acid in position 145 in SEQ ID NO: 2,
SEQ ID NO: 24 delimited by amino acid in position 76 and amino acid in position 146 in SEQ ID NO: 2,
SEQ ID NO: 26 delimited by amino acid in position 77 and amino acid in position 146 in SEQ ID NO: 2,
SEQ ID NO: 28 delimited by amino acid in position 78 and amino acid in position 146 in SEQ ID NO: 2,
SEQ ID NO: 30 delimited by amino acid in position 79 and amino acid in position 146 in SEQ ID NO: 2,
SEQ ID NO: 32 delimited by amino acid in position 80 and amino acid in position 146 in SEQ ID NO: 2,
SEQ ID NO: 34 delimited by amino acid in position 81 and amino acid in position 146 in SEQ ID NO: 2,
SEQ ID NO: 36 delimited by amino acid in position 82 and amino acid in position 146 in SEQ ID NO: 2,
SEQ ID NO: 38 delimited by amino acid in position 76 and amino acid in position 147 in SEQ ID NO: 2,
SEQ ID NO: 40 delimited by amino acid in position 77 and amino acid in position 147 in SEQ ID NO: 2,
SEQ ID NO: 42 delimited by amino acid in position 78 and amino acid in position 147 in SEQ ID NO: 2,
SEQ ID NO: 44 delimited by amino acid in position 79 and amino acid in position 147 in SEQ ID NO: 2,
SEQ ID NO: 46 delimited by amino acid in position 80 and amino acid in position 147 in SEQ ID NO: 2,
SEQ ID NO: 48 delimited by amino acid in position 81 and amino acid in position 147 in SEQ ID NO:2,
SEQ ID NO: 50 delimited by amino acid in position 82 and amino acid in position 147 in SEQ ID NO: 2,
SEQ ID NO: 52 delimited by amino acid in position 76 and amino acid in position 148 in SEQ ID NO: 2,
SEQ ID NO: 54 delimited by amino acid in position 77 and amino acid in position 148 in SEQ ID NO: 2,
SEQ ID NO: 56 delimited by amino acid in position 78 and amino acid in position 148 in SEQ ID NO: 2,
SEQ ID NO: 58 delimited by amino acid in position 79 and amino acid in position 148 in SEQ ID NO: 2,
SEQ ID NO: 60 delimited by amino acid in position 80 and amino acid in position 148 in SEQ ID NO: 2,
SEQ ID NO: 62 delimited by amino acid in position 81 and amino acid in position 148 in SEQ ID NO: 2,
SEQ ID NO: 64 delimited by amino acid in position 82 and amino acid in position 148 in SEQ ID NO: 2,
SEQ ID NO: 66 delimited by amino acid in position 76 and amino acid in position 149 in SEQ ID NO: 2,
SEQ ID NO: 68 delimited by amino acid in position 77 and amino acid in position 149 in SEQ ID NO: 2,
SEQ ID NO: 70 delimited by amino acid in position 78 and amino acid in position 149 in SEQ ID NO: 2,
SEQ ID NO: 72 delimited by amino acid in position 79 and amino acid in position 149 in SEQ ID NO: 2,
SEQ ID NO: 74 delimited by amino acid in position 80 and amino acid in position 149 in SEQ ID NO: 2,
SEQ ID NO: 76 delimited by amino acid in position 81 and amino acid in position 149 in SEQ ID NO: 2,
SEQ ID NO: 78 delimited by amino acid in position 82 and amino acid in position 149 in SEQ ID NO:2,
SEQ ID NO: 80 delimited by amino acid in position 76 and amino acid in position 150 in SEQ ID NO: 2,
SEQ ID NO: 82 delimited by amino acid in position 77 and amino acid in position 150 in SEQ ID NO: 2,
SEQ ID NO: 84 delimited by amino acid in position 78 and amino acid in position 150 in SEQ ID NO: 2,
SEQ ID NO: 86 delimited by amino acid in position 79 and amino acid in position 150 in SEQ ID NO: 2,
SEQ ID NO: 88 delimited by amino acid in position 80 and amino acid in position 150 in SEQ ID NO: 2,
SEQ ID NO: 90 delimited by amino acid in position 81 and amino acid in position 150 in SEQ ID NO: 2,
SEQ ID NO: 92 delimited by amino acid in position 82 and amino acid in position 150 in SEQ ID NO: 2,
SEQ ID NO: 94 delimited by amino acid in position 76 and amino acid in position 151 in SEQ ID NO: 2, —SEQ ID NO: 96 delimited by amino acid in position 77 and amino acid in position 151 in SEQ ID NO: 2,
SEQ ID NO: 98 delimited by amino acid in position 78 and amino acid in position 151 in SEQ ID NO: 2,
SEQ ID NO: 100 delimited by amino acid in position 79 and amino acid in position 151 in SEQ ID NO: 2,
SEQ ID NO: 102 delimited by amino acid in position 80 and amino acid in position 151 in SEQ ID NO: 2,
SEQ ID NO: 104 delimited by amino acid in position 81 and amino acid in position 151 in SEQ ID NO: 2,
SEQ ID NO: 106 delimited by amino acid in position 82 and amino acid in position 151 in SEQ ID NO: 2,
SEQ ID NO: 108 delimited by amino acid in position 76 and amino acid in position 152 in SEQ ID NO: 2,
SEQ ID NO: 110 delimited by amino acid in position 77 and amino acid in position 152 in SEQ ID NO: 2,
SEQ ID NO: 112 delimited by amino acid in position 78 and amino acid in position 152 in SEQ ID NO: 2,
SEQ ID NO: 114 delimited by amino acid in position 79 and amino acid in position 152 in SEQ ID NO: 2,
SEQ ID NO: 116 delimited by amino acid in position 80 and amino acid in position 152 in SEQ ID NO: 2,
SEQ ID NO: 118 delimited by amino acid in position 81 and amino acid in position 152 in SEQ ID NO: 2,
SEQ ID NO: 120 delimited by amino acid in position 82 and amino acid in position 152 in SEQ ID NO: 2,
SEQ ID NO: 122 delimited by amino acid in position 76 and amino acid in position 153 in SEQ ID NO: 2,
SEQ ID NO: 124 delimited by amino acid in position 77 and amino acid in position 153 in SEQ ID NO: 2,
SEQ ID NO: 126 delimited by amino acid in position 78 and amino acid in position 153 in SEQ ID NO: 2,
SEQ ID NO: 128 delimited by amino acid in position 79 and amino acid in position 153 in SEQ ID NO: 2,
SEQ ID NO: 130 delimited by amino acid in position 80 and amino acid in position 153 in SEQ ID NO: 2,
SEQ ID NO: 132 delimited by amino acid in position 81 and amino acid in position 153 in SEQ ID NO: 2,
SEQ ID NO: 134 delimited by amino acid in position 82 and amino acid in position 153 in SEQ ID NO: 2,
SEQ ID NO: 136 delimited by amino acid in position 76 and amino acid in position 154 in SEQ ID NO:2,
SEQ ID NO: 138 delimited by amino acid in position 77 and amino acid in position 154 in SEQ ID NO: 2,
SEQ ID NO: 140 delimited by amino acid in position 78 and amino acid in position 154 in SEQ ID NO: 2,
SEQ ID NO: 142 delimited by amino acid in position 79 and amino acid in position 154 in SEQ ID NO: 2,
SEQ ID NO: 144 delimited by amino acid in position 80 and amino acid in position 154 in SEQ ID NO: 2,
SEQ ID NO: 146 delimited by amino acid in position 81 and amino acid in position 154 in SEQ ID NO: 2,
SEQ ID NO: 148 delimited by amino acid in position 82 and amino acid in position 154 in SEQ ID NO:2,
SEQ ID NO: 150 delimited by amino acid in position 76 and amino acid in position 155 in SEQ ID NO: 2,
SEQ ID NO: 152 delimited by amino acid in position 77 and amino acid in position 155 in SEQ ID NO: 2,
SEQ ID NO: 154 delimited by amino acid in position 78 and amino acid in position 155 in SEQ ID NO: 2,
SEQ ID NO: 156 delimited by amino acid in position 79 and amino acid in position 155 in SEQ ID NO: 2,
SEQ ID NO: 158 delimited by amino acid in position 80 and amino acid in position 155 in SEQ ID NO: 2,
SEQ ID NO: 160 delimited by amino acid in position 81 and amino acid in position 155 in SEQ ID NO: 2,
SEQ ID NO: 162 delimited by amino acid in position 82 and amino acid in position 155 in SEQ ID NO: 2,
SEQ ID NO: 164 delimited by amino acid in position 76 and amino acid in position 156 in SEQ ID NO:2,
SEQ ID NO: 166 delimited by amino acid in position 77 and amino acid in position 156 in SEQ ID NO:2,
SEQ ID NO: 168 delimited by amino acid in position 78 and amino acid in position 156 in SEQ ID NO:2,
SEQ ID NO: 170 delimited by amino acid in position 79 and amino acid in position 156 in SEQ ID NO: 2,
SEQ ID NO: 172 delimited by amino acid in position 80 and amino acid in position 156 in SEQ ID NO: 2,
SEQ ID NO: 174 delimited by amino acid in position 81 and amino acid in position 156 in SEQ ID NO:2,
SEQ ID NO: 176 delimited by amino acid in position 82 and amino acid in position 156 in SEQ ID NO: 2,
SEQ ID NO: 178 delimited by amino acid in position 76 and amino acid in position 157 in SEQ ID NO: 2,
SEQ ID NO: 180 delimited by amino acid in position 77 and amino acid in position 157 in SEQ ID NO: 2,
SEQ ID NO: 182 delimited by amino acid in position 78 and amino acid in position 157 in SEQ ID NO: 2,
SEQ ID NO: 184 delimited by amino acid in position 79 and amino acid in position 157 in SEQ ID NO: 2,
SEQ ID NO: 186 delimited by amino acid in position 80 and amino acid in position 157 in SEQ ID NO: 2,
SEQ ID NO: 188 delimited by amino acid in position 81 and amino acid in position 157 in SEQ ID NO: 2,
SEQ ID NO: 190 delimited by amino acid in position 82 and amino acid in position 157 in SEQ ID NO:2,
SEQ ID NO: 192 delimited by amino acid in position 76 and amino acid in position 158 in SEQ ID NO: 2,
SEQ ID NO: 194 delimited by amino acid in position 77 and amino acid in position 158 in SEQ ID NO:2,
SEQ ID NO: 196 delimited by amino acid in position 78 and amino acid in position 158 in SEQ ID NO:2,
SEQ ID NO: 198 delimited by amino acid in position 79 and amino acid in position 158 in SEQ ID NO: 2,
SEQ ID NO: 200 delimited by amino acid in position 80 and amino acid in position 158 in SEQ ID NO: 2,
SEQ ID NO: 202 delimited by amino acid in position 81 and amino acid in position 158 in SEQ ID NO: 2,
SEQ ID NO: 204 delimited by amino acid in position 82 and amino acid in position 158 in SEQ ID NO: 2,
SEQ ID NO: 206 delimited by amino acid in position 76 and amino acid in position 159 in SEQ ID NO: 2,
SEQ ID NO: 208 delimited by amino acid in position 77 and amino acid in position 159 in SEQ ID NO:2,
SEQ ID NO: 210 delimited by amino acid in position 78 and amino acid in position 159 in SEQ ID NO: 2,
SEQ ID NO: 212 delimited by amino acid in position 79 and amino acid in position 159 in SEQ ID NO: 2,
SEQ ID NO: 214 delimited by amino acid in position 80 and amino acid in position 159 in SEQ ID NO: 2,
SEQ ID NO: 216 delimited by amino acid in position 81 and amino acid in position 159 in SEQ ID NO:2,
SEQ ID NO: 218 delimited by amino acid in position 82 and amino acid in position 159 in SEQ ID NO: 2,
SEQ ID NO: 220 delimited by amino acid in position 77 and amino acid in position 160 in SEQ ID NO: 2,
SEQ ID NO: 222 delimited by amino acid in position 78 and amino acid in position 160 in SEQ ID NO: 2,
SEQ ID NO: 224 delimited by amino acid in position 79 and amino acid in position 160 in SEQ ID NO: 2,
SEQ ID NO: 226 delimited by amino acid in position 80 and amino acid in position 160 in SEQ ID NO: 2,
SEQ ID NO: 228 delimited by amino acid in position 81 and amino acid in position 160 in SEQ ID NO: 2,
provided that, if present, the flanking regions of said peptidic chains in said peptide are different from the flanking regions of said sequences in SEQ ID NO: 2, or
a peptidic sequence derived from the above-defined sequence by insertion, deletion or mutation, of at least one amino acid of said peptidic chains, provided that the resulting derived sequence has a maximum length of 85 amino acids and a minimum length of 65 amino acids, and provided that said peptidic sequence is liable to inhibit DNA replication, and/or to promote cellular differentiation, or
a peptidic sequence presenting a sequence identity of at least 30% with one of the above defined sequences, provided said peptidic sequence is liable to inhibit DNA replication and/or to provide cellular differentiation,

The present invention also relates to a nucleic acid coding for one of the above-defined peptidic sequences.

The present invention also relates to a nucleic acid hybridising to a nucleic acid sequence according to claim 11, or to its complementary sequence, under the following hybridisation conditions: 6×SSC, 0.5% Sodium Dodecyl Sulfate (SDS), 65° C.

Other suitable stringent hybridization conditions can be found in Sambrook and Russel. Molecular cloning 2001 Cold Spring Harbor Laboratory Press. Cold Spring Harbor. N.Y. USA.

The present invention also relates to a nucleic acid which comprises or is constituted of at least one of the following nucleotide chains:
SEQ ID NO: 3 delimited by the nucleotide in position 226 and the nucleotide in position 480 in SEQ ID NO: 1,
SEQ ID NO: 5 delimited by the nucleotide in position 244 and the nucleotide in position 480 in SEQ ID NO: 1,
SEQ ID NO: 7 delimited by the nucleotide in position 226 and the nucleotide in position 435 in SEQ ID NO: 1,
SEQ ID NO: 9 delimited by the nucleotide in position 229 and the nucleotide in position 435 in SEQ ID NO: 1,
SEQ ID NO: 11 delimited by the nucleotide in position 232 and the nucleotide in position 435 in SEQ ID NO: 1,
SEQ ID NO: 13 delimited by the nucleotide in position 235 and the nucleotide in position 435 in SEQ ID NO: 1,
SEQ ID NO: 15 delimited by the nucleotide in position 238 and the nucleotide in position 435 in SEQ ID NO: 1,
SEQ ID NO: 17 delimited by the nucleotide in position 241 and the nucleotide in position 435 in SEQ ID NO: 1,
SEQ ID NO: 23 delimited by the nucleotide in position 226 and the nucleotide in position 438 in SEQ ID NO: 1,
SEQ ID NO: 25 delimited by the nucleotide in position 229 and the nucleotide in position 438 in SEQ ID NO: 1,
SEQ ID NO: 27 delimited by the nucleotide in position 232 and the nucleotide in position 438 in SEQ ID NO: 1,
SEQ ID NO: 29 delimited by the nucleotide in position 235 and the nucleotide in position 438 in SEQ ID NO: 1,
SEQ ID NO: 31 delimited by the nucleotide in position 238 and the nucleotide in position 438 in SEQ ID NO: 1,
SEQ ID NO: 33 delimited by the nucleotide in position 241 and the nucleotide in position 438 in SEQ ID NO: 1,
SEQ ID NO: 35 delimited by the nucleotide in position 244 and the nucleotide in position 438 in SEQ ID NO: 1,
SEQ ID NO: 37 delimited by the nucleotide in position 226 and the nucleotide in position 441 in SEQ ID NO: 1,
SEQ ID NO: 39 delimited by the nucleotide in position 229 and the nucleotide in position 441 in SEQ ID NO: 1,
SEQ ID NO: 41 delimited by the nucleotide in position 232 and the nucleotide in position 441 in SEQ ID NO: 1,
SEQ ID NO: 43 delimited by the nucleotide in position 235 and the nucleotide in position 441 in SEQ ID NO: 1,
SEQ ID NO: 45 delimited by the nucleotide in position 238 and the nucleotide in position 441 in SEQ ID NO: 1,
SEQ ID NO: 47 delimited by the nucleotide in position 241 and the nucleotide in position 441 in SEQ ID NO: 1,
SEQ ID NO: 49 delimited by the nucleotide in position 244 and the nucleotide in position 441 in SEQ ID NO: 1,
SEQ ID NO: 51 delimited by the nucleotide in position 226 and the nucleotide in position 444 in SEQ ID NO: 1,
SEQ ID NO: 53 delimited by the nucleotide in position 229 and the nucleotide in position 444 in SEQ ID NO: 1,
SEQ ID NO: 55 delimited by the nucleotide in position 232 and the nucleotide in position 444 in SEQ ID NO: 1,
SEQ ID NO: 57 delimited by the nucleotide in position 235 and the nucleotide in position 444 in SEQ ID NO: 1,
SEQ ID NO: 59 delimited by the nucleotide in position 238 and the nucleotide in position 444 in SEQ ID NO: 1,
SEQ ID NO: 61 delimited by the nucleotide in position 241 and the nucleotide in position 444 in SEQ ID NO: 1,
SEQ ID NO: 63 delimited by the nucleotide in position 244 and the nucleotide in position 444 in SEQ ID NO: 1,
SEQ ID NO: 65 delimited by the nucleotide in position 226 and the nucleotide in position 447 in SEQ ID NO: 1,
SEQ ID NO: 67 delimited by the nucleotide in position 229 and the nucleotide in position 447 in SEQ ID NO: 1,
SEQ ID NO: 69 delimited by the nucleotide in position 232 and the nucleotide in position 447 in SEQ ID NO: 1,
SEQ ID NO: 71 delimited by the nucleotide in position 235 and the nucleotide in position 447 in SEQ ID NO: 1,
SEQ ID NO: 73 delimited by the nucleotide in position 238 and the nucleotide in position 447 in SEQ ID NO: 1,
SEQ ID NO: 75 delimited by the nucleotide in position 241 and the nucleotide in position 447 in SEQ ID NO: 1,
SEQ ID NO: 77 delimited by the nucleotide in position 244 and the nucleotide in position 447 in SEQ ID NO: 1,
SEQ ID NO: 79 delimited by the nucleotide in position 226 and the nucleotide in position 450 in SEQ ID NO: 1,
SEQ ID NO: 81 delimited by the nucleotide in position 229 and the nucleotide in position 450 in SEQ ID NO: 1, —SEQ ID NO: 83 delimited by the nucleotide in position 232 and the nucleotide in position 450 in SEQ ID NO: 1,
SEQ ID NO: 85 delimited by the nucleotide in position 235 and the nucleotide in position 450 in SEQ ID NO: 1,
SEQ ID NO: 87 delimited by the nucleotide in position 238 and the nucleotide in position 450 in SEQ ID NO: 1,
SEQ ID NO: 89 delimited by the nucleotide in position 241 and the nucleotide in position 450 in SEQ ID NO: 1,
SEQ ID NO: 91 delimited by the nucleotide in position 244 and the nucleotide in position 450 in SEQ ID NO: 1,
SEQ ID NO: 93 delimited by the nucleotide in position 226 and the nucleotide in position 453 in SEQ ID NO: 1,
SEQ ID NO: 95 delimited by the nucleotide in position 229 and the nucleotide in position 453 in SEQ ID NO: 1,
SEQ ID NO: 97 delimited by the nucleotide in position 232 and the nucleotide in position 453 in SEQ ID NO: 1,
SEQ ID NO: 99 delimited by the nucleotide in position 235 and the nucleotide in position 453 in SEQ ID NO: 1,
SEQ ID NO: 101 delimited by the nucleotide in position 238 and the nucleotide in position 453 in SEQ ID NO: 1,
SEQ ID NO: 103 delimited by the nucleotide in position 241 and the nucleotide in position 453 in SEQ ID NO: 1,
SEQ ID NO: 105 delimited by the nucleotide in position 244 and the nucleotide in position 453 in SEQ ID NO: 1,
SEQ ID NO: 107 delimited by the nucleotide in position 226 and the nucleotide in position 456 in SEQ ID NO: 1,
SEQ ID NO: 109 delimited by the nucleotide in position 229 and the nucleotide in position 456 in SEQ ID NO: 1,
SEQ ID NO: 11 delimited by the nucleotide in position 232 and the nucleotide in position 456 in SEQ ID NO: 1,
SEQ ID NO: 113 delimited by the nucleotide in position 235 and the nucleotide in position 456 in SEQ ID NO: 1,
SEQ ID NO: 115 delimited by the nucleotide in position 238 and the nucleotide in position 456 in SEQ ID NO: 1,
SEQ ID NO: 117 delimited by the nucleotide in position 241 and the nucleotide in position 456 in SEQ ID NO: 1,
SEQ ID NO: 119 delimited by the nucleotide in position 244 and the nucleotide in position 456 in SEQ ID NO: 1,
SEQ ID NO: 121 delimited by the nucleotide in position 226 and the nucleotide in position 459 in SEQ ID NO: 1,
SEQ ID NO: 123 delimited by the nucleotide in position 229 and the nucleotide in position 459 in SEQ ID NO: 1,
SEQ ID NO: 125 delimited by the nucleotide in position 232 and the nucleotide in position 459 in SEQ ID NO: 1,
SEQ ID NO: 127 delimited by the nucleotide in position 235 and the nucleotide in position 459 in SEQ ID NO: 1,
SEQ ID NO: 129 delimited by the nucleotide in position 238 and the nucleotide in position 459 in SEQ ID NO: 1,
SEQ ID NO: 131 delimited by the nucleotide in position 241 and the nucleotide in position 459 in SEQ ID NO: 1,
SEQ ID NO: 133 delimited by the nucleotide in position 244 and the nucleotide in position 459 in SEQ ID NO: 1,
SEQ ID NO: 135 delimited by the nucleotide in position 226 and the nucleotide in position 462 in SEQ ID NO: 1,
SEQ ID NO: 137 delimited by the nucleotide in position 229 and the nucleotide in position 462 in SEQ ID NO: 1, —SEQ ID NO: 139 delimited by the nucleotide in position 232 and the nucleotide in position 462 in SEQ ID NO: 1,
SEQ ID NO: 141 delimited by the nucleotide in position 235 and the nucleotide in position 462 in SEQ ID NO: 1,
SEQ ID NO: 143 delimited by the nucleotide in position 238 and the nucleotide in position 462 in SEQ ID NO: 1,
SEQ ID NO: 145 delimited by the nucleotide in position 241 and the nucleotide in position 462 in SEQ ID NO: 1,
SEQ ID NO: 147 delimited by the nucleotide in position 244 and the nucleotide in position 462 in SEQ ID NO: 1,
SEQ ID NO: 149 delimited by the nucleotide in position 226 and the nucleotide in position 465 in SEQ ID NO: 1, —SEQ ID NO: 151 delimited by the nucleotide in position 229 and the nucleotide in position 465 in SEQ ID NO: 1,
SEQ ID NO: 153 delimited by the nucleotide in position 232 and the nucleotide in position 465 in SEQ ID NO: 1,
SEQ ID NO: 155 delimited by the nucleotide in position 235 and the nucleotide in position 465 in SEQ ID NO: 1,
SEQ ID NO: 157 delimited by the nucleotide in position 238 and the nucleotide in position 465 in SEQ ID NO: 1,
SEQ ID NO: 159 delimited by the nucleotide in position 241 and the nucleotide in position 465 in SEQ ID NO: 1,
SEQ ID NO: 161 delimited by the nucleotide in position 244 and the nucleotide in position 465 in SEQ ID NO: 1,
SEQ ID NO: 163 delimited by the nucleotide in position 226 and the nucleotide in position 468 in SEQ ID NO: 1,
SEQ ID NO: 165 delimited by the nucleotide in position 229 and the nucleotide in position 468 in SEQ ID NO: 1,
SEQ ID NO: 167 delimited by the nucleotide in position 232 and the nucleotide in position 468 in SEQ ID NO: 1,
SEQ ID NO: 169 delimited by the nucleotide in position 235 and the nucleotide in position 468 in SEQ ID NO: 1,
SEQ ID NO: 171 delimited by the nucleotide in position 238 and the nucleotide in position 468 in SEQ ID NO: 1,
SEQ ID NO: 173 delimited by the nucleotide in position 241 and the nucleotide in position 468 in SEQ ID NO: 1,
SEQ ID NO: 175 delimited by the nucleotide in position 244 and the nucleotide in position 468 in SEQ ID NO: 1,
SEQ ID NO: 177 delimited by the nucleotide in position 226 and the nucleotide in position 471 in SEQ ID NO: 1,
SEQ ID NO: 179 delimited by the nucleotide in position 229 and the nucleotide in position 471 in SEQ ID NO: 1,
SEQ ID NO: 181 delimited by the nucleotide in position 232 and the nucleotide in position 471 in SEQ ID NO: 1,
SEQ ID NO: 183 delimited by the nucleotide in position 235 and the nucleotide in position 471 in SEQ ID NO: 1,
SEQ ID NO: 185 delimited by the nucleotide in position 238 and the nucleotide in position 471 in SEQ ID NO: 1,
SEQ ID NO: 187 delimited by the nucleotide in position 241 and the nucleotide in position 471 in SEQ ID NO: 1,
SEQ ID NO: 189 delimited by the nucleotide in position 244 and the nucleotide in position 471 in SEQ ID NO: 1,
SEQ ID NO: 191 delimited by the nucleotide in position 226 and the nucleotide in position 474 in SEQ ID NO: 1,
SEQ ID NO: 193 delimited by the nucleotide in position 229 and the nucleotide in position 474 in SEQ ID NO: 1,
SEQ ID NO: 195 delimited by the nucleotide in position 232 and the nucleotide in position 474 in SEQ ID NO: 1,
SEQ ID NO: 197 delimited by the nucleotide in position 235 and the nucleotide in position 474 in SEQ ID NO: 1,
SEQ ID NO: 199 delimited by the nucleotide in position 238 and the nucleotide in position 474 in SEQ ID NO: 1,
SEQ ID NO: 201 delimited by the nucleotide in position 241 and the nucleotide in position 474 in SEQ ID NO: 1,
SEQ ID NO: 203 delimited by the nucleotide in position 244 and the nucleotide in position 474 in SEQ ID NO: 1,
SEQ ID NO: 205 delimited by the nucleotide in position 226 and the nucleotide in position 477 in SEQ ID NO: 1,
SEQ ID NO: 207 delimited by the nucleotide in position 229 and the nucleotide in position 477 in SEQ ID NO: 1,
SEQ ID NO: 209 delimited by the nucleotide in position 232 and the nucleotide in position 477 in SEQ ID NO: 1,
SEQ ID NO: 211 delimited by the nucleotide in position 235 and the nucleotide in position 477 in SEQ ID NO: 1,
SEQ ID NO: 213 delimited by the nucleotide in position 238 and the nucleotide in position 477 in SEQ ID NO: 1,
SEQ ID NO: 215 delimited by the nucleotide in position 241 and the nucleotide in position 477 in SEQ ID NO: 1,
SEQ ID NO: 217 delimited by the nucleotide in position 244 and the nucleotide in position 477 in SEQ ID NO: 1,
SEQ ID NO: 219 delimited by the nucleotide in position 229 and the nucleotide in position 480 in SEQ ID NO: 1,
SEQ ID NO: 221 delimited by the nucleotide in position 232 and the nucleotide in position 480 in SEQ ID NO: 1,
SEQ ID NO: 223 delimited by the nucleotide in position 235 and the nucleotide in position 480 in SEQ ID NO: 1,
SEQ ID NO: 225 delimited by the nucleotide in position 238 and the nucleotide in position 480 in SEQ ID NO: 1,
SEQ ID NO: 227 delimited by the nucleotide in position 241 and the nucleotide in position 480 in SEQ ID NO: 1,
provided that, if present, the flanking regions of said nucleotide chains in said nucleic acid are different from the flanking regions of said nucleotide chains in SEQ ID NO: 1, or
a nucleic acid sequence derived from the above-defined sequence by insertion, deletion or mutation of at least one nucleotide in said nucleotide chains, provided that the resulting derived nucleic acid sequence has a maximum length of 255 nucleotides and a minimum length of 195 nucleotides, and provided that said derived nucleic acid codes for a peptidic sequence liable to inhibit DNA replication and/or to promote cellular differentiation, or
a nucleic acid presenting a sequence identity of at least 33% with one of the above-defined sequences, provided that said nucleic acid sequence codes for a peptidic sequence liable to inhibit DNA replication and/or to promote cellular differentiation or its complementary sequence,
or the complementary sequence of one of the above-defined nucleic acid sequences,
or an antisense of the above-defined nucleic acid sequences.

The present invention also relates to a eukaryotic or prokaryotic expression vector comprising a nucleic acid such as defined above, and the elements necessary for its expression in a eukaryotic or a prokaryotic cell.

In a preferred embodiment of the invention, the above-mentioned eukaryotic or prokaryotic cell is transformed by a nucleic acid such as defined above, or by a vector such as defined above.

The present invention also relates to a polyclonal or monoclonal antibody, directed against a peptidic sequence such as defined above.

The present invention also relates to an idiotypic antibody directed against the paratope of the above-defined antibody.

The present invention also relates to a method for screening drugs liable to enhance DNA replication, in cells, comprising the following steps:
contacting a peptidic sequence as defined above with a compound to screen,
selecting the compounds which bind to said peptidic sequence,
optionally checking that the selected compounds enhance DNA replication.

The present invention also relates to a method for screening drugs liable to enhance DNA replications comprising the following steps:
contacting a peptidic sequence as defined above with a compound to screen and with a ligand of said peptidic sequence, such as an antibody, a scFv polypeptide, an aptamer, or the Cdt1 protein,
selecting the compounds which prevent the binding of the ligand to said peptidic sequence, and which do not bind to said ligand,
optionally checking that the selected compounds enhance DNA replication.

The procedures for the preparation of the above mentioned antibodies scFv polypeptides or aptamers are particularly well known to the man skilled in the art.

The present invention also relates to a method for screening drugs liable to inhibit DNA replication comprising the following steps:
contacting the Cdt1 protein and a peptidic sequence as defined above with a compound to screen,
selecting the compounds which prevent binding of Cdt1 to said peptidic sequence, and which do not bind to said peptidic sequence,
optionally checking that the selecting compounds inhibit DNA replication.

### DESCRIPTION OF THE FIGURES

FIG. 1A, FIG. 1B and FIG. 1C

FIG. 1A represents the functional domain organization of Geminin. The numbering is for human Geminin (adapted from 48). The star symbol (*) indicates phosphorylated sites, Ser45 and Ser49⁵. From left to right, the destruction box (23-31); the neutralization domain (28-79) and the replication inhibition domain (76-160). Overlap between domains is shown by mixed colors. The coiled-coil (LZ) domain is indicated by the back dashed area, 110-144.

FIG. 1B represents the Vertebrate Geminin sequences alignment of the DNA replication inhibition domain, according to positions 79 to 160 in human Geminin sequence HsGem (SwissProt accession number O75496) (SEQ ID NO: 2), Xenopus laevis XlGem (heavy form: SwissProt accession number O93352; light form: SwissProt accession number O93355), mouse MmGem (SwissProt accession number O88513), zebra fish DrGem (MGC accession number AAH55552) and Rattus norvegicus RnGem (RefSeq accession number XP 214477). Letters above the sequences indicate the heptad repeat ‘a,b,c,d,e,f,g’ positions assigned according the crystal structure. Arrows indicate limits of the HsGeminin deletion mutants in FIG. 3.

FIG. 1C gives a helical wheel representation of the repeated sequence of the HsGem-LZ highlighting the ‘a’ and ‘d’ positions, relative numbering according to the peptide sequence, Leu2 in the peptide corresponding to Leu110 in the HsGem sequence. Residues at the acidic ‘g’ position are in italic.

FIG. 2A, FIG. 2B and FIG. 2C

FIG. 2A represents the overall structure of HsGem-LZ peptide (L2-A37) in Cα trace representation. The two monomers form a parallel coiled-coil. The alternating layers of ‘a’ and ‘d’ residues are displayed as stick models in the center, in light and dark color respectively. Electrostatic pairing between ‘e’ and ‘g’ positions are outlined in dark colors at the periphery (K27, H13, E22 and E8).

FIG. 2B represents a ribbon diagram of the view in FIG. 2A rotated 90° along the two-fold axis.

FIG. 2C represents an electrostatic potential surface computed with the program GRASP 49.

FIG. 3A, FIG. 3B and FIG. 3C

FIG. 3A represents the activity of HsGeminin deletion mutants. The DNA synthesis inhibition activity of a series of HsGeminin deletion mutant proteins was measured by incubation in Xenopus egg extracts. Inhibition of DNA synthesis by the wild type protein was observed at concentration between 75 and 100 nM, while maximal inhibition of DNA synthesis by the peptides was observed at a concentration of 250 nM. DNA synthesis was measured by incorporation of a radioactive label DNA precursor (dCTP) upon 2 hours incubation at room temperature. Numbers indicate the amino-acids of the HsGeminin protein. LZ=coiled-coil domain.

FIG. 3B represents a coomassie blue staining of fractions eluted from a sucrose gradient loaded with HsGem-N80 mutant and resolved by SDS-PAGE. Arrows indicated the position of the molecular weight standards.

FIG. 3C represents a scan of the SDS-PAGE of FIG. 3B. D=dimer; Tri=trimer; Tet=tetramer.

FIG. 4A, FIG. 4B, FIG. 4C and FIG. 4D

FIG. 4A represents far ultraviolet (UV) CD spectra of HsGem-LZ peptide as a function of temperature in 12 mM NaPi and 20 mM NaCl (pH 6.1). The thick trace was recorded at 25° C. Peptide concentration was 48 μM.

FIG. 4B represents far UV CD spectra of HsGem-LZ peptide as a function of pH in 12 mM NaPi and 20 mM NaCl at 5° C.

FIG. 4C represents the molar ellipticity 0222 of HsGem-LZ peptide as a function of pH in 12 mM NaPi at the various indicated temperatures (° C.).

FIG. 4D represents the pH dependence of the molar ellipticity Θ222 of HsGem-LZ peptide as a function of temperature at 20 mM NaCl and 150 mM NaCl.Tm were extracted and plotted versus pH (inset).

FIG. 5

FIG. 5 represents a comparison of NMR spectra of HsGem-LZ and HsGem(82-145). The spectra of HsGem(82-145) were recorded at 22° C. and 32° C. (top spectrum) and the concentration was 7 mg/ml. For the spectrum labeled “1/10 HsGem(82-145)” the concentration was 0.7 mg/ml.

FIG. 6A, FIG. 6B, FIG. 6C and FIG. 6D

FIG. 6A represents the experimental data (crosses) for HsGem-LZ superimposed with the scattering curve (dashed line) computed from the crystal atomic model by CRYSOL (χ²=0.98).

FIG. 6B represents experimental data (crosses) for HsGem(82-145) superimposed with the scattering curve (dashed line) computed by CRYSOL from the proposed model fitted in the average GASBOR models (χ²=2.02).

FIGS. 6C and 6D respectively represent the pair distance distribution functions P(r) (crosses) for HsGem-LZ and HsGem(82-145) Geminin constructs computed from X-ray scattering curves with the program GNOM. The P(r) function computed by GASBOR for real space fitted ab initio models are represented as dashed lines.

FIG. 7A and FIG. 7B

FIG. 7A represents the low resolution shape of HsGem-LZ model (two orthogonal views) obtained from SAXS data, represented as a semi-transparent surface and superimposed on the X-ray structure shown as a colored stick model.

FIG. 7B represents the low resolution shape of HsGem(82-145) presented as a semi-transparent surface with dimensions of approximately 11.4×4.3×4.2 nm. The superimposed model is shown with two dimers. Each dimer comprises the experimental coiled-coil domain of five heptads and a dummy model (almost globular in shape) corresponding to the N-terminal extensions of 29 residues.

### EXAMPLES

### Example 1

### Determination of the Crystal Structure of the Geminin-LZ

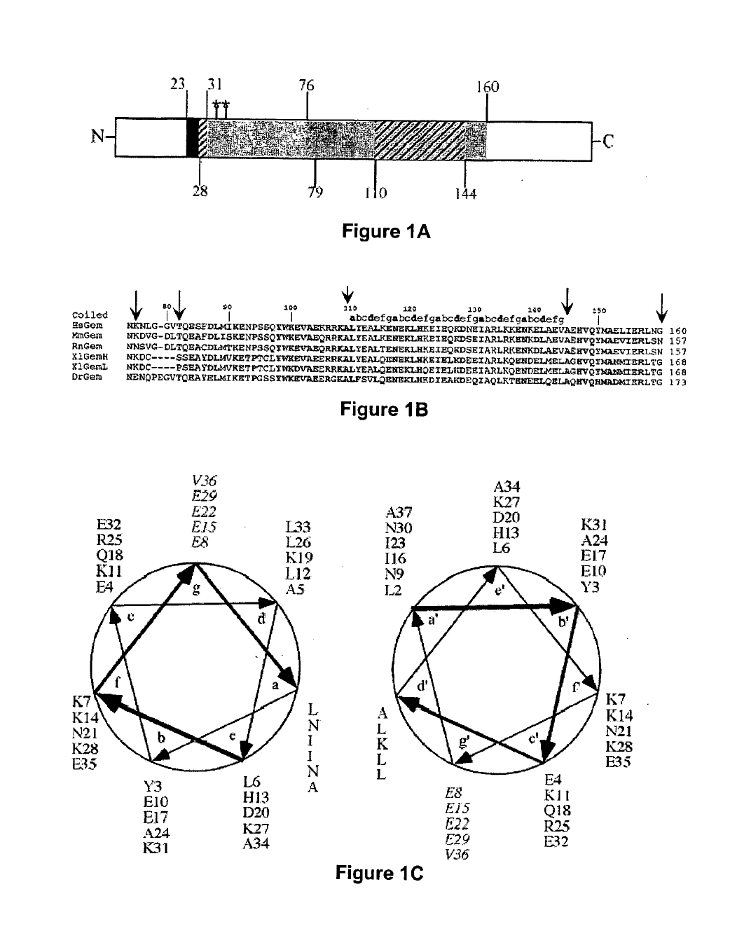

A peptide corresponding to the predicted coiled-coil fragment (residues 110-145) of human Geminin capped by a N-terminal Thr residue (FIG. 1A) was produced by standard peptide synthesis and crystallized as previously reported¹³.

Briefly, The HsGem-LZ peptide comprising residues Leu110-Ala145 of human Geminin with an extra N-terminal capping Thr residue was synthesized by Fmoc solid-phase peptide synthesis and purified by HPLC in acetonitrile/water¹³. Crystals were prepared by hanging drop technique using a peptide solution of 26 mg/ml and a reservoir containing 100 mM Hepes buffer at pH 7.5, 10% PEG 6K and 5% MPD. Crystals were transferred in a cryo-protective solution supplemented at 20% MPD and flash cooled at 100° K before data collection. Diffraction images with oscillation angles of 0.5° were recorded using a MarResearch area detector mounted on a Rigaku rotating anode generator operating at 40 kV 90 mA and equipped with Osmic mirrors. The high and low resolution data corresponding to a total oscillation range of 112° and 163°, respectively, were processed using the programs MOSFLM and SCALA³⁶. The statistics for data collection at 1.47 Å resolution are summarized in Table 1.

The crystal structure contains a dimer in the asymmetric unit and was determined by molecular replacement. Molecular replacement was implemented by the program EPMR³⁷. The search models were various parallel or anti-parallel dimeric coiled-coils as well as trimeric or tetrameric coiled-coils. A unique well contrasted solution using diffraction data between 10 and 3.5 Å was found for dimeric parallel two-stranded coiled-coil (1E7T) that yielded a correlation coefficient of 0.49 and a Reryst of 0.44. The phases were improved and extended to higher resolution by a few rounds of solvent flattening with histogram matching using DM³⁸. Most of the Geminin side chains could be clearly identified in the resulting idealized electron density map and alternate conformations were found for side-chains of glutamics A10 and B35. Iterations of refinement steps and manual fitting used the programs REFMAC³⁹ and O⁴⁰. Final refinement statistics are summarized in Table 1.

The Inventors found a unique solution involving a parallel, two-stranded coiled-coil. The structure refined at 1.47 Å resolution contains 74 residues and 125 water molecules, and has R-factor and free R-factor of 17.9 and 22.1 respectively (Table 1).

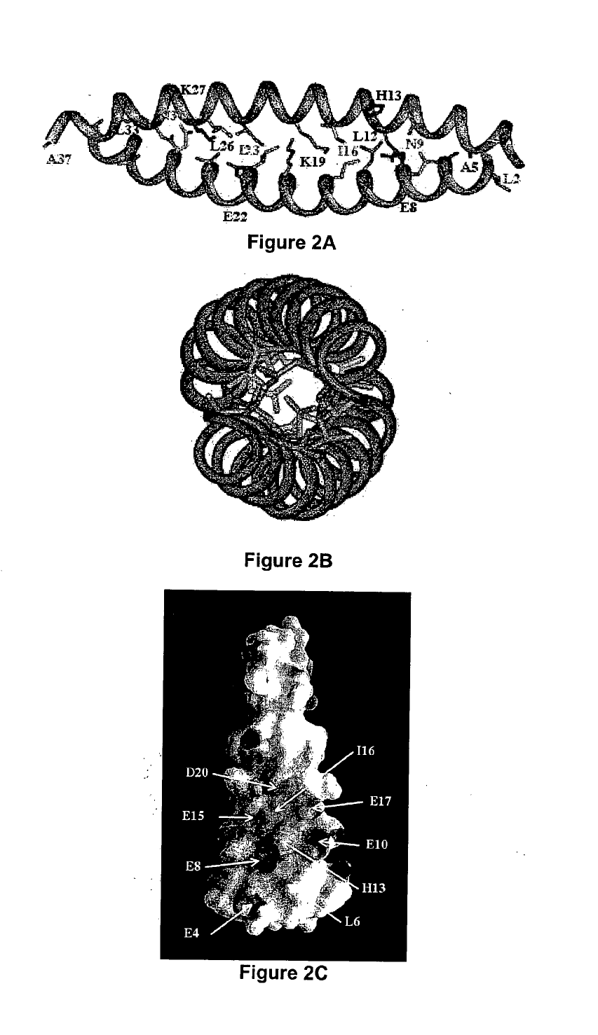

The structure of the HsGem-LZ peptide is a parallel homodimer coiled-coil with a length of about 60 Å and a diameter of about 20 Å. The canonical α-helical structure of each segment comprises residues 110 to 145 (FIG. 1A). This value is in agreement with circular dichroism experiments showing a very high content of α-helical structure (see below). The helices display canonical ‘knobs-into-holes’ packing 1415, in which the side chains at the ‘a’ and ‘d’ positions of heptad repeat motifs form successive layers (FIGS. 2A and 2B). Every side chain inserts into the hole formed by four residues on the opposite helix. This inter-twined packing arrangement corresponds the classical packing mode observed in GCN4 and Fos-Jun Leucine zippers^{16; 17}.

The distance between the helical axis ranges from 8.9 Å to 10.3 Å, from the edge to the center, respectively. Mean rise per residue in helices A and B is about 1.53 Å and the number of residues by α-helical turn about 3.64 Å, a value more closely related to a regular α-helix (3.6) than to a classical coiled-coil (3.5). The two helices in the HsGem-LZ adopt similar main-chain conformations. The rmsd difference for the 37 Cα atoms is 0.44 Å and the local symmetry axis corresponds to a classical dyad axis. The rotation angle is however 167.4° and induces a small but significant distortion of symmetrical arrangement of the helices. The largest rmsd for main chain atoms (0.6 to 0.8 Å) are observed for 3 residues in the middle of the helix and for the N and C-terminal residues.

It could be verified that helix capping by the N-terminal Thr residue (substituting an Ala in the wild type HsGem sequence) contributes efficiently to the stabilization of the helix. Each N-cap contains an identical well-defined network of hydrogen bonds and hydratation patterns. The Oγ atom of Thr1 makes an H-bond to the main-chain NH of Glu4 and the carbonyl group of Thr1 donates a forked H-bond to the NHs of Glu4 and Ala5. The NH of Thr1 makes an H-bond to the side-chain carboxyl group of Glu4.

The coordinates and structure factors are deposited in the RCSB Protein Data Bank under the accession code 1T6F.

TABLE 1
X-Ray data collection statistics and refinement of the HsGem-LZ crystal

| space group | P2₁2₁2₁ |
|---|---|
| unit cell | a = 25.12, b = 43.44, |
| parameters (Å) | c = 67.50 |
| wavelength (Å) | 1.5418 |
| number of measurements | 53,623 |
| number of unique | 12,976 |
| reflections | |
| resolution range (Å) | 25.0-1.47 (1.47-1.59) |
| completeness (%) | 92.5 (87.9) |
| average intensity I/σ(I) | 18.0 (3.9) |
| R_{merge}* | 0.056 (0.276) |
| number of protein atoms | 627 |
| number of waters | 125 |
| number reflections | 12,976/595 |
| (working/free) | |
| Rwork/Rfree (%)§ | 17.9/22.1 |
| Overall B-factor (Å2) | 17.8 |
| Average B for main | 14.4 |
| chain atoms | |
| Average B for side | 17.3 |
| chain atoms | |
| Average B for | 28.3 |
| solvent atoms | |
| R.m.s.d. from ideal | |
| geometry | |
| bond lengths (Å) | 0.009 |
| bond angles (°) | 1.142 |
| Ramachandran | |
| plot (%)** | |
| Most favored region | 100. |

numbers in parentheses refer to reflections in the outer resolution shell.
*R_{merge} = Σₕ|Iᵢ(h) · <I(h)>|/ΣI(h) where <I(h)> is the average intensity of equivalent reflections Iᵢ(h) and the sum is extended over all measured observations for all unique reflections.
**Procheck⁴⁷
§Rwork = Σ|F^{o}_{h,k,l} − F^{c}_{h,k,l}|/Σ|F^{o}_{h,k,l}| where F^{o}_{h,k,l} and F^{c}_{h,k,l} are the observed and calculated structure factors amplitudes.

The monomers associate into a dimer through the formation of an extensive interface which buries 11% (2187 Å²) of the accessible surface area of each monomer. The dimer is predominantly stabilized by hydrophobic interactions. The interface involves 70% of non-polar and 30% of polar residues, nine hydrogen bonds, three bridging water molecules but no salt bridge. Most of the residues at ‘a’ and ‘d’ positions of the five heptad repeats (FIGS. 1B and 2A)—including Leu2, Ala5, Leu12, Ile16, Lys19, Ile23, Leu26 and Leu33, are hydrophobic and pack in a typical ‘knobs in holes’ mode 15. As expected in parallel coiled-coil, the classical hydrogen bonded ion pairs occur at ‘g’ and succeeding ‘e’ positions occurs in HsGem-LZ for two positions (over four possible) between Glu8 and His13′, and Glu22 and Lys27′ (FIG. 2A).

The analysis of charge properties and conservation on the surface of the coiled-coil domain may help to gain further insight into possible interaction sites (FIG. 2D). An acidic patch is found which involves residues Glu8, Glu10, Glu15, Glu17 and Asp20. Exposed within this acidic surface of 944 Å² are found the strictly conserved residues Leu6 and His13. As shown in FIG. 2C this surface is mostly negatively charged but displays also hydrophobic and polar residues. In the crystal, residues Glu10 and Glu17 in this acidic surface make contacts with residues Asn21 and Arg25 from another molecule.

Circular dichroism experiments were then recorded with the HsGem-LZ peptide in order to investigate its thermal and pH stability, and to compare with other coiled-coils.

Briefly, CD Spectra were recorded on a JASCO-810 spectrometer equipped with a temperature controller and 0.1 cm path length cuvettes. Spectra were recorded in 0.2 nm steps from 260 to 195 nm with an integration time of 0.5 sec at each wavelength, and the baseline corrected against a cuvette containing buffer alone. Spectra were recorded from 1° C. to 60° C., at various pH from 2.6 to 8.3, and NaCl concentrations (20, 100 and 150 mM).

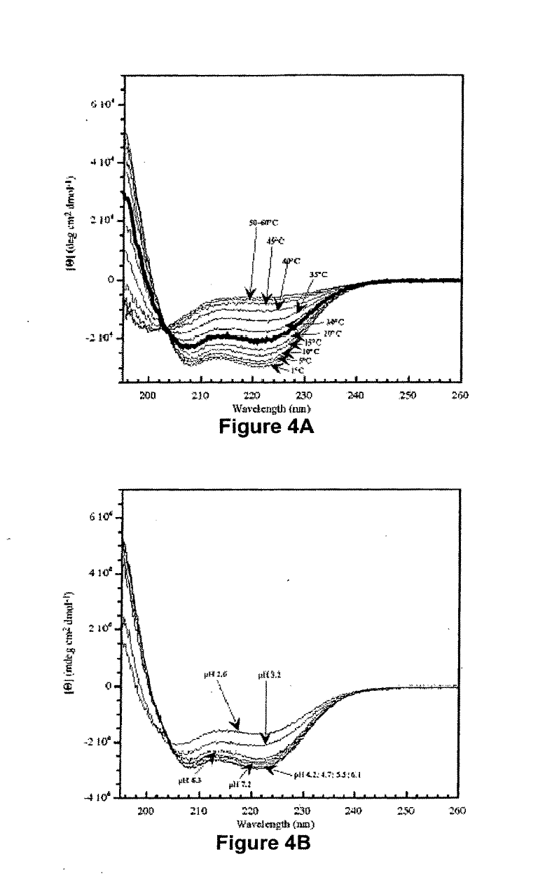

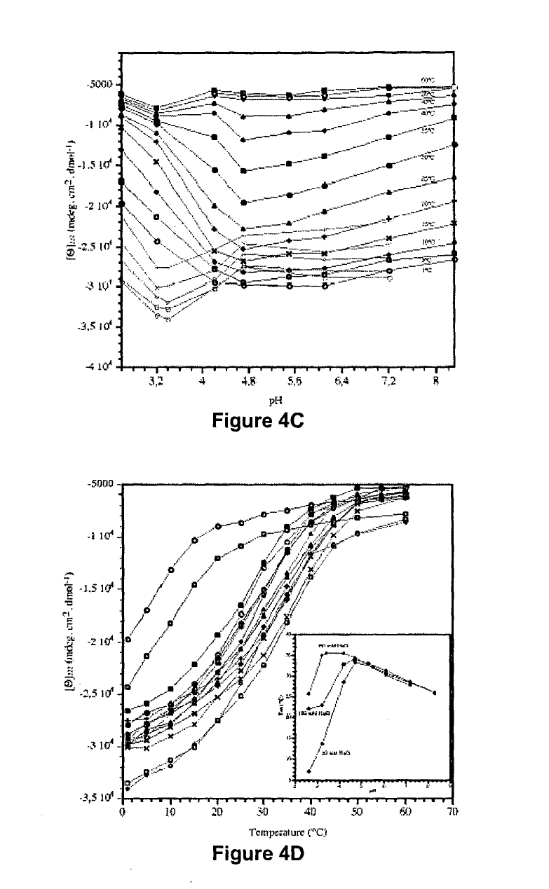

No significant irreversibility of CD spectra was detected when temperature, pH and salt concentration was cycled. The thick trace in FIG. 4A shows the CD spectrum of the GemH LZ in 20 mM NaCl (pH 6.1) at 25° C. This spectrum represents 50% random coil structure (50% helical). On lowering the temperature to 1° C. (FIG. 4A, bottom trace), the helical content is increased to 80% with the appearance of minima near 222 nm and 208 nm. The value of the Θ222/Θ208 ratio for non-coiled helices is typically near 0.83 and increases to about 1.03 in coiled-coils 20. The Θ222/Θ208 ratio for HsGem-LZ is 1.02. The isodichroic point near 203 nm is an evidence of a two-state transition 21 between unstructured and the coiled-coil structured peptide.

The data shown in FIG. 4B illustrate the pH behavior at 20 mM NaCl, with a strong CD signal for pH between 4.2 and 6.1, whereas at acidic pH (2.6 and 3.2) the signal is small. Plots of Θ222 versus pH at the 13 different temperatures are shown in FIG. 4C. The maximum ellipticity is observed at pH 2.6-3.2 and 150 mM NaCl. At low ionic strength (20 mM NaCl) maximum ellipticity is observed at pH 5.5-6.1. Comparison of the data recorded at 20 mM and 150 mM NaCl (FIG. 4C) indicates different behavior according to salt concentration and pH.

Melting temperature (Tm) values extracted from the curves of FIG. 4D are plotted in the inset versus pH at the three ionic strengths. Similar curves are found for pH values above 4.7, probably indicating that the conformation of HsGem-LZ is not sensitive to ionic strength for pH above 4.7. At more acidic pH values, a dramatic loss of stability is observed at low ionic strength (20 mM NaCl) and α-helical content of HsGem-LZ is almost undetectable at room temperature in such conditions. The HsGem-LZ forms stable coiled-coil at acidic pH and high ionic strength. The pH and salt dependences shown in FIGS. 4C-4D support the importance of charge-charge interactions in the folding pathway of the HsGem-LZ peptide^{20; 21}.

The Tm values for coiled-coils of similar size than HsGem-LZ are found in the range of 40° C. to 70° C.^{22; 23}, as compared to HsGem-LZ which has a Tm of 35° C. This indicates that the HsGem-LZ domain is less stable compared to other coiled-coils. Analysis of sequence partnering and specificity of the DNA replication inhibitory region of Geminins (FIG. 1B) from various species indicates a high conservation inside the heptad repeats of the Geminin coiled-coil. Hydrophobic interacting positions at the coiled-coil interface in the dimer follow the scheme LNIILNA (with two Asn) for the ‘a’ position and ALKLL (with one Lys) for the ‘d’ position in the heptads. Asn is quite common at the ‘a’ position, and its presence has been correlated with the occurrence of a parallel dimeric state¹⁷. The Gem-LZ sequences have an unexpected high occurrence of polar residues (two Asn and one Lys) in ‘a’ or ‘d’ positions, and accordingly suggests that Gem-LZ must form homodimers in vivo. Asn and Lys residues using a coiled-coil pattern in the PIR data base were searched for, to define how specific is the Gem-LZ. Among the 19 retrieved sequences, 10 give a good score with the COILS program, but only 6 sequences have at least 5 heptads, which all belong to Geminin sequences. This indicate that Geminin has a rather unique coiled-coil pattern, with three polar residues in either the ‘a’ or ‘d’ positions. The bZIP specificity prediction program²⁴, was used to compare the coiled-coil partnering specificity of Geminin homodimers against other coiled-coils. Geminin homodimer gave systematically the highest score, supporting that Geminin may form homodimers in the cell.

### Example 2

### Low Resolution Shape of Geminin Coiled-Coil Containing Domain

The inventors' observation of Geminin oligomers with the HsGem(80-212) protein, which keep the ability of Geminin to inhibit DNA synthesis (see below), led them to investigate its three-dimensional structure. However, the attempts using this construct were unsuccessful. Thus, it was decided to use smaller Geminin constructs encompassing the coiled-coil region. SAXS and NMR experiments were performed with HsGem(82-145) and HsGem-LZ (as a control) to analyze their oligomerization status and to derive their low resolution structures.

Briefly, the ¹H-NMR spectra of HsGem(82-145) in 20 mM Tris-HCl, pH=8.0 and 100 mM NaCl (90% H₂O; 10% D₂O) were recorded on a 500 MHz Bruker Avance spectrometer equipped with a cryo-probe.

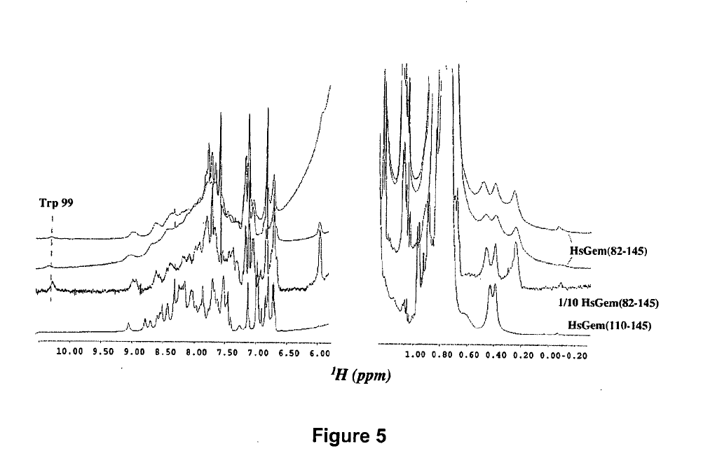

FIG. 5 illustrates the differences of linewidth between the NMR spectra of HsGem coiled-coil containing peptides. Linewidth of a given NMR signal, is at first approximation related to correlation time τc. The enlargement of the molecular weight will induce an increase of τc and thus broadening the linewidth. The up-field shifted signals (CδH₃ of Leu2) of HsGem-LZ at 0.4 ppm are used for comparison with the corresponding signals of HsGem(82-145). As shown in FIG. 5, a slight increase of the linewidth is observed between HsGem-LZ and the low concentration sample of HsGem(82-145) (0.7 mg/ml), compatible with the difference of molecular weights between HsGem(82-145) and HsGem-LZ. The linewidth of the HsGem(82-145) signals is concentration dependant and is dramatically increased in spectra recorded at 7 mg/ml concentrations. Accordingly, this indicates that HsGem(82-145) forms oligomers.

Recent progresses in SAXS methodology provide new structural tools to explore biological macromolecules. SAXS experiments were recorded for the HsGem(82-145) protein at concentrations of 4 and 8 mg/ml.

Briefly, the two Geminin samples HsGem-LZ and HsGem82-145 were prepared by dialyzing the purified protein solutions in 20 mM Tris-HCl buffer at pH 8.0 and 100 mM NaCl. The synchrotron radiation SAXS data were collected following standard procedures on the D24 beam line on the storage ring DCI of LURE (Orsay, France) using a linear detector. The sample-detector distance was 163.1 cm. This enabled a scattering magnitude range of 0.004 Å⁻¹<s<0.046 Å⁻¹ to be covered with s=2 sin θ/λ where 2θ is the scattering angle and λ=1.488 Å the wavelength of the x-ray radiation. The scattering profiles were collected at 8° C. in eight successive 100 seconds frames. Judging from the stability of intensity versus time, there was no radiation damage of protein samples during data collection. Background measurements were performed with buffer solutions. The data were normalized to the intensity of the incident beam corrected for the detector response; the scattering of the buffer was subtracted. The radii of gyration R_{g} and forward scattering intensity I(0) were evaluated by the Guinier approximation with the program PRIMUS⁴¹. The distance distribution function, P(r), shows the frequency of vector r, relating any two volume elements within the entire volume of the scattering particle. It was calculated using the indirect Fourier transform method implemented in the GNOM program⁴² and provided the maximum particle dimension, Dₘₐₓ. The I(0) and R_{g} values were also obtained from the zero^{th} and the second moment of the P(r) function, respectively. The forward scattering intensity I(0) is related to the protein molecular weight M_{w} by equation 1:

I(0)=κ(ρₚ−ρₛ)²v²cM_{w}/Nₐ (1)

where κ is an experimental constant, vₚ the partial specific volume, c the concentration in mg/mL, Nₐ the Avogadro number, ρₚ and ρ_{c} the average electron density of protein and solvent, respectively⁴³. The molecular masses of the two Geminin solutes were evaluated by calibration against reference solutions of chicken egg white lysozyme (M_{w}=14,300 Daltons) and Xenopus Laevis Mob1 (M_{w}=23,300 Daltons). Low-resolution shapes of Geminin were restored from the scattering intensity profiles of monodisperse solution of these proteins using the ab initio procedure GASBOR²⁵. A 2-fold symmetry was assumed in both cases with 35 and 64 residues/monomer corresponding to the primary sequence of these two proteins, HsGem-LZ and HsGem(82-145), respectively. The uniqueness and the stability of the restored envelopes were checked by repeating the minimization. About 10 independent models were aligned and averaged with the programs SUPCOMB⁴⁴ and DAMAVER⁴⁵ to build a representative model. The scattering profiles from the atomic models were calculated using the program CRYSOL⁴⁶. Default parameters were used and the solvent density values (0.35 e−/Å³ for HsGem-LZ and 0.355 e−/Å³ for HsGem(82-145)) were adjusted to achieve the best fits.

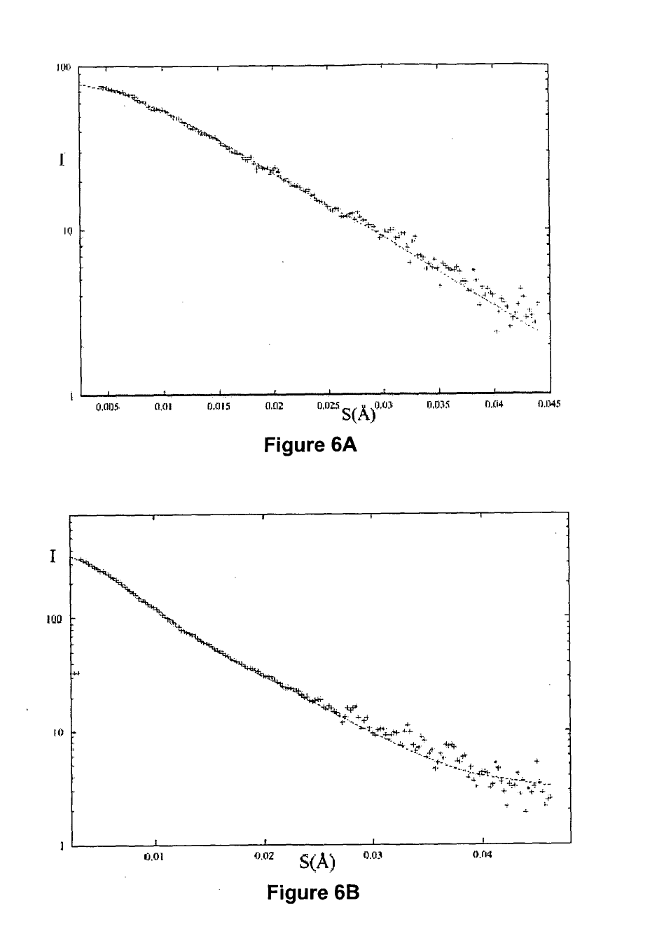

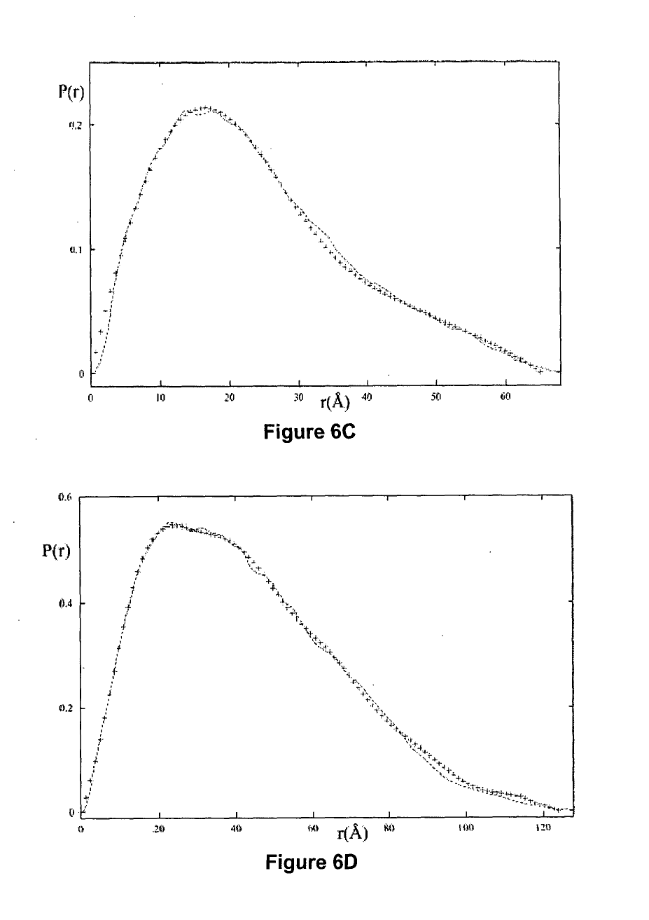

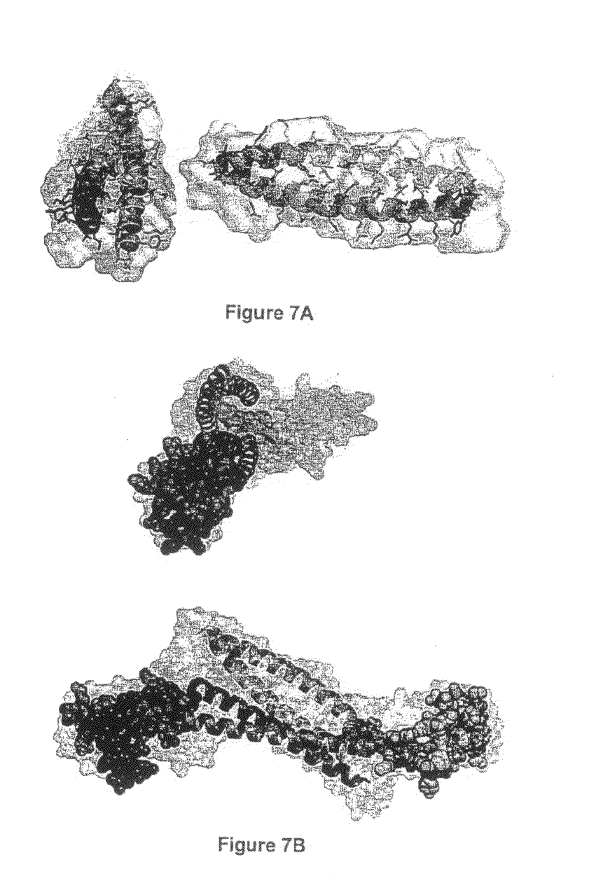

Thus, a radius of gyration R_{g}=3.5±0.1 nm and a maximal dimension Dₘₐₓ of 12±1 nm were obtained from the Guinier plot and from the pair distribution function P(r) (FIG. 6), respectively. The R_{g} and I(0) values from the P(r) function agreed well with those derived from the Guinier plots. Similar experiments were recorded with HsGem-LZ, and the structural parameters for the isolated coiled-coil domain of Geminin, give a R_{g} of 2.0±0.06 mm and the P(r) yielded a maximum dimension Dₘₐₓ of 6.5±0.5 nm (FIG. 6). The zero extrapolation I(0) of each profile is proportional to the molecular mass of the scattering particle and is compared to the forward scattering data of two reference proteins (lysozyme and Mob1) collected at the same period. The obtained data yield the average molecular weights of 7.5 kDa and 35.9 kDa for HsGem-LZ and for HsGem(82-145), respectively. Comparison between these MW estimates and the monomer molecular weight calculated from the corresponding amino-acid composition (4.3 and 7.7 kDa, respectively) clearly establish that HsGem-LZ is a dimer and HsGem(82-145) is a tetramer, in the range of concentrations used. The SAXS data obtained for HsGem(82-145) are typical of an elongated protein: (i) the molecule has a large R_{g} for a protein of this molecular weight (32 kDa) and (ii) the profile of P(r), revealing the histogram of interatomic distances within this particle, is spread with a maximal dimension Dₘₐₓ of 12 nm. The average ab initio low resolution shape of HsGem-LZ obtained by simulated annealing program GASBOR²⁵ is shown in FIG. 7A. The X-ray structure of the dimeric HsGem-LZ coiled-coil has been fitted within the ab initio envelope represented by the spatial distribution of dummy residues and shows an excellent agreement (FIG. 7A). This is consistent with the comparison between the theoretical scattering curve of HsGem-LZ calculated by CRYSOL using the crystal coordinates and the experimental scattering profile (FIG. 6A). The low resolution shape of HsGem(82-145) protein was obtained using the same procedure, starting from the P(r) function. The average shape of the dummy residues model is illustrated in FIG. 7B superimposed with a putative model build by first fitting the two parallel coiled-coil domains (HsGem-LZ) within the central region of the low resolution envelope, avoiding steric clashes. It was assumed then, that the 29-residues N-terminal domain extensions have a compact elongated conformation that can easily be docked in the low resolution ab initio model. The resulting structure of HsGem(82-145) constructed in this way is a tetramer with overall dimensions of 11.4×4.3×4.2 nm, constituted by two dimeric parallel coiled-coil assembled ‘head-to-tail’ in an antiparallel fashion. The scattering curve from this model was computed by CRYSOL (FIG. 6B) and yields a good fit to the experimental SAXS data (discrepancy index χ²=2.0). The low resolution of the data (˜2 nm) is sufficient to distinguish between the N-terminal domains and the more elongated coiled-coil segments. Moreover these data revealed that additional intermolecular interactions occur in the tetramer between coiled-coils and N-terminal domains.

### Example 3

### DNA Replication Inhibition Activity of Geminin Coiled-Coil

In order to determine whether a homodimer form of Geminin is functional, the Inventors have tested the ability of this peptide to inhibit DNA synthesis in an in vitro DNA replication assay derived from Xenopus eggs¹⁸.

Briefly, Xenopus egg extracts were prepared as previously described³⁵. Inhibition assays were carried out in a 20 μl reaction containing 3 ng/μl of sperm nuclei and the indicated amounts of proteins at 1:40 ratio (protein to extract). Replication was measured by incorporation of α³²P dCTP following 2 hours incubation at room temperature.

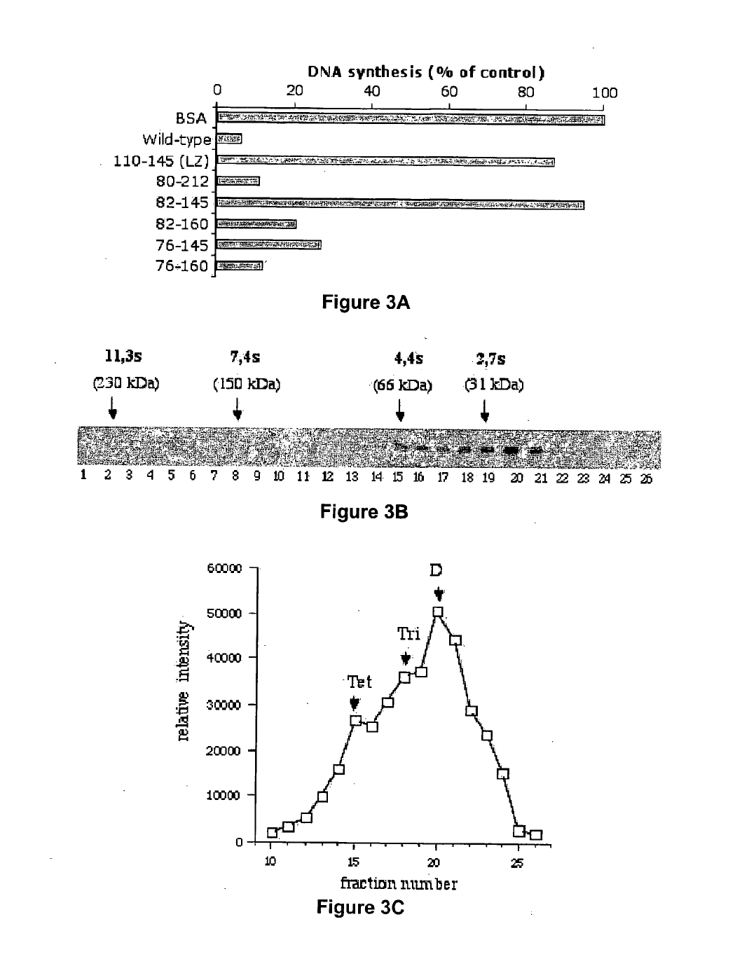

This system has been previously shown to be appropriate to determine the activity of HsGeminin². Interestingly the peptide containing the coiled-coil domain of HsGeminin did not interfere with DNA synthesis even at concentration much higher than those of the wild-type protein (e.g.: 320 nM). This latter efficiently inhibited DNA synthesis, compared to a control reaction with a non-specific protein (BSA, FIG. 3A). This result shows that although the coiled-coil is necessary for both Geminin functions⁶, as well as Geminin dimerization¹³, it is not sufficient to inhibit DNA synthesis.

This result prompted the Inventors to investigate with more accuracy the amino-acids requirements for Geminin inhibition. They produced a set of peptides containing additional amino-acids of the HsGeminin protein either at the N-terminal or at C-terminal of the coiled-coil domain.

Deletion mutants of Human Geminin were made by PCR amplification (Master Mix Qiagen) and insertion into pET15(b) between the Nde1 and BamH1 sites. The sequences of the primers used to generate each construct were: ggaattccatatgaaaaatcttggaggagtcacc (SEQ ID NO: 20) or ggaattccatatgacccaggagtcatttgatctt (SEQ ID NO: 21) for sequence starting at 76 or 82, respectively, and cgggatccttatgctacttctgccagttcttt (SEQ ID NO: 22) or cgggatccttaaccattcagtctctctattag (SEQ ID NO: 229) for sequences ending at residue 145 or 160, respectively. The DNA sequence of each insert was confirmed after enzymatic cleavage and sequencing (ABI PRISM 310 Genetic Analyzer). These hexahistidine-tagged proteins were expressed in E. coli strain BL21-DE3 and purified according to standard protocols (Qiagen). Proteins were dialyzed against 10 mM Tris-HCl pH 8, 300 mM NaCl at 4° C. before use. The His-tag of the HsGem82-145 protein was removed by thrombin cleavage and the protein was further purified by exclusion chromatography.

The mutant 82-145, which contains a four-basic residues stretch (RKKR; see FIG. 1A) at the N-terminus compared to the coiled-coil (110-145 mutant), was also ineffective in inhibiting DNA synthesis. The mutant 82-160, which contains 15 amino-acids more at the C-terminus of HsGeminin inhibited DNA synthesis, although less efficiently compared to the wild-type protein. These results suggest that extra sequences in the C-terminal extension of the coiled-coil region of Geminin are important for its function. The other mutants tested, in which amino-acids from 76 to 145 were present, were also effective in inhibiting DNA synthesis. Taken together these results suggest that the coiled-coil itself is not functional but that both carboxy- and amino-terminal residues (amino-acids between 76 and 160) are necessary for Geminin function. The Inventors further tested whether a form of HsGeminin (residues 80-212) that contains the coiled-coil domain plus the entire carboxy-terminal part of the protein could form dimers. This protein was effective in inhibiting DNA synthesis (FIG. 3A) and was fractionated by a sucrose gradient centrifugation.

Briefly, purified HsGemininN80 mutant protein (50 μg) was diluted to 0.140 ml with XB buffer (100 mM KCl, 2 mM MgCl₂, 0.1 mM CaCl₂, 10 mM Hepes-KOH pH 7.7, 50 mM sucrose) and loaded onto a linear 5 to 20% sucrose gradient made in XB. A mix of protein standards was run in parallel. Gradients were run at 40 000 rpm in a SW55Ti rotor for 20 hours at 4° C. Fractions were collected from the bottom of the tube and analyzed by SDS-PAGE followed by staining with Coomassie blue. The intensity of the signals was determined with the ImageQuant software.

FIG. 3B shows that this protein has a broad sedimentation profile ranging from about 25 to 90 kDa, with a major peak at 30 kDa. Scanning of the signals shows that discrete peaks corresponding to apparent mass of 42.5 and 66 kDa are present. Assuming a globular shape, these could correspond to a trimer and a tetramer of this form of HsGeminin (the size of one monomer being 14.9 kDa). However, as Geminin has an asymmetric form 19, the broad range of sedimentation of Geminin may corresponds to oligomers more than tetramers.

The Inventors have shown that the coiled-coil domain alone is not sufficient to inhibit DNA synthesis and that sequence extensions at the N- and C-terminus of the coiled-coil are required to give a functional domain. Full inhibition, comparable to the wild-type Geminin, is obtained with the HsGem(80-212) and HsGem(76-160) proteins. The proteins HsGem(76-145) and HsGem(82-160) are 70 to 80% as efficient, while HsGem(82-145) is not functional. In HsGem(76-145) the six residues added at the N-terminus are not conserved in Geminin sequences (FIG. 1B). Interestingly, adding 15 residues at the C-terminus of HsGem(82-145) rescues DNA synthesis inhibition. These extra residues are conserved and predicted to form a helix, not involved in the coiled-coil itself, but most probably protruding at the C-terminus of the coiled-coil. This rescue of function by adding residues, at either N- or C-terminus of the non-functional HsGem(82-145) sequence may be the result of structure stabilization. This effect of either N- or C-terminal sequences to the non-functional HsGem-LZ is in agreement with the tetrameric structure proposed for the HsGem(82-145) protein, where the dimers interact “head to tail” (see below) and these added residues on either sides are relatively close in space.

Circular dichroism indicated that charge-charge interactions are important for the stability of Geminin-LZ homodimers and that they can form in physiological conditions. The SAXS data suggest that HsGem(82-145) self-associate to form a tetramer in solution and that the two homodimers are associated in a “head to tail” orientation. Circular dichroism experiments demonstrated the low thermal stability of the HsGem-LZ peptide compared to other coiled-coils, and provide evidences of the equilibrium between unfolded peptide and the coiled-coil structure.

The Inventors then defined the modalities of the interaction between this coiled-coil region and its effectors. First, the concentration dependence of the HsGem-LZ quaternary structure may play a role. Higher Geminin concentrations in the cell could lead to a higher proportion of dimerization and tetramerization. Indeed, over-expression of Geminin does enhance the potency of replication inhibition in cells where Geminin is normally expressed at lower levels 6 Second, other regions of the Geminin molecule may influence the conformation of the coiled-coil domain. For instance, the modeling data suggest that the N-terminal regions of two Geminin molecules may interact in a manner insufficient to provide the driving force for dimerization in the absence of the LZ region, but with sufficient affinity to stabilize the coiled-coil. Alternatively, the N-terminal region may form a surface that favors and therefore stabilizes the folded conformation of the LZ region. Third, effector molecules may be attracted to the unstructured C-terminal tail of Geminin, and the LZ may be induced to fold only after complexation, or as part of the binding event. Examples of induced fold have been observed in many types of proteins, including those involved in transcriptional activation^{26; 27}, RNA binding^{28; 29} and cell-cycle progression^{30; 31}. To date, interactions have been detected between the Geminin and the CDT1 proteins in Xenopus egg extracts^{1; 3} or using recombinant protein technology in mammals³². Recently, it has been shown that CDT1 phosphorylation by cyclin A-dependent kinases plays a crucial role in negative regulation of its function after S phase³³ although it does not affect the binding to Geminin. These results suggest two regulation pathways involving CDT1: one Geminin-dependent and the other cycline A-kinase dependent. The CDT1-Geminin complex can form at replication origins but this complex does not inhibit replication¹⁹. The Geminin-dependent inhibition of DNA replication requires its accumulation on chromatin, possibly through oligomerization of Geminin. It is known that CDT1 binding to Geminin involves the coiled-coil region of Geminin⁶ and Geminin oligomerization may possibly may affect this binding. Geminin also binds to basic-residues-rich sequences of Hox proteins, and interactions compete with CDT1 binding⁸. Similarly, interaction has also been reported between Geminin and the differentiation factor Six-3. The Inventors observed a patch of acidic residues (FIG. 2C), which potentially may interact with basic residues of a partner-protein (CDT1 and Hox proteins).

Other regions of Geminin, upstream and downstream sequences from the coiled-coil domain are required for its function. Several basic residues (including many conserved residues in FIG. 1) are found in the N-terminal extension of about 30 residues in length. Interestingly, such domain organization is similar to the one observed in the bZIP family of transcription factors, i.e. myc, max, fos, jun³⁴. While Geminin-LZ region resembles those of the bZIP family, no direct DNA interaction has been reported for Geminin. The Inventors used homology modeling to build the structure of Gem-LZ in interaction with DNA taking the Fos-Jun-DNA complex structure (LFOS) as a template. Basic residues of Geminin fit well for interacting with phosphate groups of the DNA, as in the Fos-Jun-DNA complex. However, as expected, several hydrophobic or bulky residues of Geminin (Tyr98, Trp99 and Val 102) have steric clashes with the DNA bases. This simple modeling strategy shows that Geminin would not be able to bind a regular dsDNA without distortion of the DNA base pairing. The structure formed by the DNA molecule at replication origins is not known, but logically would rather resemble to a unwound piece of DNA, and expectedly with missing DNA base-pairing. Thus, Geminin would preferably bind to these DNA segments occurring in replication origins.

Geminin is a regulatory protein found in metazoans, but is apparently missing from yeast genomes. Geminin appears to be involved not solely in DNA replication regulation but also in cellular differentiation processes 7; 8. Subsequently, oligomerization might be as well involved in the differentiation function of Geminin. In these aspects, the Inventors' crystal structure of Geminin dimerization domain provides a rational for designing drugs able to compete with or stabilize the Geminin coiled-coil oligomers.

### REFERENCES

1. Tada, S., L1, A., Maiorano, D., Mechali, M. & Blow, J. J. (2001). Repression of origin assembly in metaphase depends on inhibition of RLF-B/Cdt1 by geminin. Nat Cell Biol 3, 107-13.
2. Wohlschlegel, J. A., Dwyer, B. T., Dhar, S. K., Cvetic, C., Walter, J. C. & Dutta, A. (2000). Inhibition of eukaryotic DNA replication by geminin binding to Cdt1. Science 290, 2309-12.
3. Maiorano, D., Moreau, J. & Mechali, M. (2000). XCDT1 is required for the assembly of pre-replicative complexes in Xenopus laevis. Nature 404, 622-5.
4. Rialland, M., Sola, F. & Santocanale, C. (2002). Essential role of human CDT1 in DNA replication and chromatin licensing. J Cell Sci 115, 1435-40.
5. Kulartz, M., Kreitz, S., Hiller, E., Damoc, E. C., Przybylski, M. & Knippers, R. (2003). Expression and phosphorylation of the replication regulator protein geminin. Biochem Biophys Res Commun 305, 412-20.
6. McGarry, T. J. & Kirschner, M. W. (1998). Geminin, an inhibitor of DNA replication, is degraded during mitosis. Cell 93, 1043-53.
7. Del Bene, F., Tessmar-Raible, K. & Wittbrodt, J. (2004). Direct interaction of geminin and Six3 in eye development. Nature 427, 745-9.
8. Luo, L., Yang, X., Takihara, Y., Knoetgen, H. & Kessel, M. (2004). The cell-cycle regulator geminin inhibits Hox function through direct and polycomb-mediated interactions. Nature 427, 749-53.
9. Kroll, K. L., Salic, A. N., Evans, L. M. & Kirschner, M. W. (1998). Geminin, a neuralizing molecule that demarcates the future neural plate at the onset of gastrulation. Development 125, 3247-58.
10. Wolf, E., Kim, P. S. & Berger, B. (1997). MultiCoil: a program for predicting two- and three-stranded coiled coils. Protein Sci 6, 1179-89.
11. Cohen, C. & Parry, D. A. (1994). Alpha-helical coiled coils: more facts and better predictions. Science 263, 488-9.
12. Uversky, V. N., Gillespie, J. R. & Fink, A. L. (2000). Why are “natively unfolded” proteins unstructured under physiologic conditions? Proteins 41, 415-27.
13. Thepaut, M., Hoh, F., Dumas, C., Calas, B., Strub, M. P. & Padilla, A. (2002). Crystallization and preliminary X-ray crystallographic analysis of human Geminin coiled-coil domain. Biochim Biophys Acta 1599, 149-51.
14. Crick, F. H. C. (1953). The packing of α-helices: simple coiled-coils. Acta Cryst. 6, 689-697.
15. Walther, D., Eisenhaber, F. & Argos, P. (1996). Principles of helix-helix packing in proteins: the helical lattice superposition model. J Mol Biol 255, 536-53.
16. Vieth, M., Kolinski, A. & Skolnick, J. (1996). Method for predicting the state of association of discretized protein models. Application to leucine zippers. Biochemistry 35, 955-67.
17. Zeng, X., Herndon, A. M. & Hu, J. C. (1997). Buried asparagines determine the dimerization specificities of leucine zipper mutants. Proc Natl Acad Sci USA 94, 3673-8.
18. Blow, J. J. & Laskey, R. A. (1986). Initiation of DNA replication in nuclei and purified DNA by a cell-free extract of Xenopus eggs. Cell 47, 577-87.
19. Maiorano, D., Rul, W. & Mechali, M. (2004). Cell cycle regulation of the licensing activity of Cdt1 in Xenopus laevis. Exp Cell Res 295, 138-149.
20. Zhou, N. E., Kay, C. M. & Hodges, R. S. (1994). The net energetic contribution of interhelical electrostatic attractions to coiled-coil stability. Protein Eng 7, 1365-72.
21. Jelesarov, I., Durr, E., Thomas, R. M. & Bosshard, H. R. (1998). Salt effects on hydrophobic interaction and charge screening in the folding of a negatively charged peptide to a coiled coil (leucine zipper). Biochemistry 37, 7539-50.
22. Akey, D. L., Malashkevich, V. N. & Kim, P. S. (2001). Buried polar residues in coiled-coil interfaces. Biochemistry 40, 6352-60.
23. Newman, J. R. & Keating, A. E. (2003). Comprehensive identification of human bZIP interactions with coiled-coil arrays. Science 300, 2097-101.
24. Fong, J. H., Keating, A. E. & Singh, M. (2004). Predicting specificity in bZIP coiled-coil protein interactions. Genome Biol 5, R11.
25. Svergun, D. I., Petoukhov, M. V. & Koch, M. H. (2001). Determination of domain structure of proteins from X-ray solution scattering. Biophys J 80, 2946-53.
26. Kussie, P. H., Gorina, S., Marechal, V., Elenbaas, B., Moreau, J., Levine, A. J. & Pavletich, N. P. (1996). Structure of the MDM2 oncoprotein bound to the p53 tumor suppressor transactivation domain. Science 274, 948-53.
27. Radhakrishnan, I., Perez-Alvarado, G. C., Parker, D., Dyson, H. J., Montminy, M. R. & Wright, P. E. (1997). Solution structure of the KIX domain of CBP bound to the transactivation domain of CREB: a model for activator:coactivator interactions. Cell 91, 741-52.
28. Battiste, J. L., Mao, H., Rao, N. S., Tan, R., Muhandiran, D. R., Kay, L. E., Frankel, A. D. & Williamson, J. R. (1996). Alpha helix-RNA major groove recognition in an HIV-1 rev peptide-RRE RNA complex. Science 273, 1547-51.
29. Puglisi, J. D., Chen, L., Blanchard, S. & Frankel, A. D. (1995). Solution structure of a bovine immunodeficiency virus Tat-TAR peptide-RNA complex. Science 270, 1200-3.
30. Kriwacki, R. W., Hengst, L., Tennant, L., Reed, S. I. & Wright, P. E. (1996). Structural studies of p21Waf1/Cip1/Sdi1 in the free and Cdk2-bound state: conformational disorder mediates binding diversity. Proc Natl Acad Sci USA 93, 11504-9.
31. Pavletich, N. P. (1999). Mechanisms of cyclin-dependent kinase regulation: structures of Cdks, their cyclin activators, and Cip and INK4 inhibitors. J Mol Biol 287, 821-8.
32. Yanagi, K., Mizuno, T., You, Z. & Hanaoka, F. (2002). Mouse geminin inhibits not only Cdt1-MCM6 interactions but also a novel intrinsic Cdt1 DNA binding activity. J Biol Chem 277, 40871-80.
33. Sugimoto, N., Tatsumi, Y., Tsurumi, T., Matsukage, A., Kiyono, T., Nishitani, H. & Fujita, M. (2004). Cdt1 phosphorylation by cyclin A-dependent kinases negatively regulates its function without affecting geminin binding. J Biol Chem.
34. Luscombe, N. M., Austin, S. E., Berman, H. M. & Thornton, J. M. (2000). An overview of the structures of protein-DNA complexes. Genome Biol 1, REVIEWS001.
35. Menut, S., Lemaitre, J., Hair, A. & Méchali, M. (1988). DNA replication and chromatin assembly using Xenopus egg extracts. In Advances in Molecular Biology: A comparative Methods Approach to the Study of Ooocytes and Embryos (Richter, J. D., ed.). Oxford University Press, Oxford.
36. CCP4. (1994). The CCP4 Suite: Programs for Protein Crystallography. Acta Cryst. D50, 760-763.
37. Kissinger, C. R., Gehlhaar, D. K. & Fogel, D. B. (1999). Rapid automated molecular replacement by evolutionary search. Acta Crystallogr D Biol Crystallogr 55 (Pt 2), 484-91.
38. Cowtan, K. (1999). Error estimation and bias correction in phase-improvement calculations. Acta Crystallogr D Biol Crystallogr 55 (Pt 9), 1555-67.
39. Murshudov, G. N., Vagin, A. A. & Dodson, E. J. (1997). Refinement of Macromolecular Structures by the Maximum-Likelihood Method. Acta Cryst. D53, 240-255.
40. Jones, T. A., Zou, J.-Y., Cowan, S. W. & Kljeldgaard, M. (1991). Improved methods for building protein models in electron density maps and the location of errors in these models. Acta Cryst. A47, 110-119.
41. Konarev, P. V., Volkov, V. V., Sokolova, A. V., Koch, M. H. & Svergun, D. I. (2003). PRIMUS: a Windows PC-based system for small-angle scattering data analysis. J. Appl. Cryst. 36, 1277-1282.
42. Svergun, D. I. (1992). Determination of the regularization parameter in indirect-transform methods using perceptual criteria. J Appl Cryst 25, 495-503.
43. Glatter, O. (1982). Data treatment. In Small Angle X-Ray Scattering (O. Glatter, a. O. K., ed.), pp. 119-196. Academic Press, London, London.
44. Kozin, M. B. & Svergun, D. I. (2001). Automated matching of high- and low-resolution structural models. J Appl Cryst 34, 33-41.
45. Volkov, V. V. & Svergun, D. I (2003). Uniqueness of ab initio shape determination in small-angle scattering. J Appl Cryst 36, 860-864.
46. Svergun, D. I., Barberato, C. & Koch, M. H. J. (1995), CRYSOL—a program to evaluate X-ray solution scattering of biological macromolecules from atomic coordinates. J Appl Cryst 28, 768-773.
47. Laskowski, R. A., Moss, D. S. & Thornton, J. M. (1993). Main-chain bond lengths and bond angles in protein structures. J Mol Biol 231, 1049-67.
48. Quinn, L. M., Herr, A., McGarry, T. J. & Richardson, H. (2001). The Drosophila Geminin homolog: roles for Geminin in limiting DNA replication, in anaphase and in neurogenesis. Genes Dev 15, 2741-54.
49. Nicholls, A., Sharp, K. A. & Honig, B. (1991). Protein folding and association: insights from the interfacial and thermodynamic properties of hydrocarbons. Proteins 11, 281-96.

1-20. (canceled)

21. A method for treating pathologies implying pathological DNA replication and/or differentiation disorders, or disturbance of the cellular proliferation/differentiation balance,
a peptidic sequence which comprises or is constituted of a peptidic chain of at least 65 contiguous amino acids selected from the amino acid sequence SEQ ID NO: 4, SEQ ID NO: 4 being delimited by the amino acid in position 76 and by the amino acid in position 160 of SEQ ID NO: 2, provided that, if present, the flanking regions of said peptidic chain in said peptidic sequence are different from the flanking regions of said peptidic chain in SEQ ID NO: 2, or
a peptidic sequence derived from the above-defined peptidic sequence by insertion, deletion or substitution of at least one amino acid in said peptidic chain, provided that the resulting derived peptidic chain has a maximum length of 85 amino acids and a minimum length of 65 amino acids, and provided that said peptidic sequence is liable to inhibit DNA replication, and/or to promote cellular differentiation, or
a peptidic sequence presenting a sequence identity of at least 30% with one of the above defined sequences, provided that said peptidic sequence is liable to inhibit DNA replication and/or to provide cellular differentiation, said peptidic sequences being optionally in the form of a dimer, or
an antibody directed to at least one of said peptidic sequences, or
a nucleic acid sequence coding for one at least of said peptidic sequences, or its complementary sequences.

22. The method according to claim 21, wherein the peptidic sequence, is such that the amino acids corresponding or homologous to the amino acids in positions 106, 109, 110, 112, 113, 114, 116, 118, 121, 123, 124, 125, and 128, of SEQ ID NO: 2 are not mutated.

23. The method of a peptidic sequence according to claim 21 the subject has a disease involving pathological cell proliferation, or involving impaired cell differentiation such as developmental abnormalities.

24. The method according to claim 21, wherein said antibody or said nucleic acid is administered to treat apopoptosis, Parkinson's disease, Alzheimer disease, multiple sclerosis, spinal cord injury, cellular dedifferentiation, autism, mental retardation, or a vascular lesion formation.

25. A pharmaceutical composition, comprising as active substance:
a peptidic sequence which comprises or is constituted of a peptidic chain of at least 65 contiguous amino acids selected from the amino acid sequence SEQ ID NO: 4, SEQ ID NO: 4 being delimited by the amino acid in position 76 and by the amino acid in position 160 of SEQ ID NO: 2, provided that, if present, the flanking regions of said peptidic chain in said peptidic sequence are different from the flanking regions of said peptidic chain in SEQ ID NO: 2, or
a peptidic sequence derived from the above-defined peptidic sequence by insertion, deletion or substitution of at least one amino acid in said peptidic chain, provided that the resulting derived peptidic chain has a maximum length of 85 amino acids and a minimum length of 65 amino acids, and provided that said peptidic sequence is liable to inhibit DNA replication, and/or to promote cellular differentiation, the resulting derived peptidic sequence being in particular such that the amino acids corresponding or homologous to the amino acids in positions 106, 109, 110, 112, 113, 114, 116, 118, 121, 123, 124, 125, and 128, of SEQ ID NO: 2 are not mutated, or
a peptidic sequence presenting a sequence identity of at least 30% with one of the above-defined sequences, provided that said peptidic sequence is liable to inhibit DNA replication and/or to provide cellular differentiation, said peptidic sequences being optionally in the form of a dimmer, or
an antibody directed against one of said sequences,
in association with a pharmaceutically acceptable vehicle.

26. A pharmaceutical composition according to claim 25, wherein the peptidic sequence comprises or is constituted of one of the following amino acid chains:
SEQ ID NO: 4 delimited by amino acid in position 76 and amino acid in position 160 in SEQ ID NO: 2,
SEQ ID NO: 6 delimited by amino acid in position 82 and amino acid in position 160 in SEQ ID NO: 2,
SEQ ID NO: 8 delimited by amino acid in position 76 and amino acid in position 145 in SEQ ID NO: 2,
SEQ ID NO: 10 delimited by amino acid in position 77 and amino acid in position 145 in SEQ ID NO: 2,
SEQ ID NO: 12 delimited by amino acid in position 78 and amino acid in position 145 in SEQ ID NO: 2,
SEQ ID NO: 14 delimited by amino acid in position 79 and amino acid in position 145 in SEQ ID NO: 2,
SEQ ID NO: 16 delimited by amino acid in position 80 and amino acid in position 145 in SEQ ID NO: 2,
SEQ ID NO: 18 delimited by amino acid in position 81 and amino acid in position 145 in SEQ ID NO: 2,
provided that, if present, the flanking regions of said sequences are different from the flanking regions of said sequences in SEQ ID NO: 2, or
a peptidic sequence derived from the above-defined peptidic sequence by insertion, deletion or mutation, of at least one amino acid in said peptidic chains, provided that the resulting derived sequence has a maximum length of 85 amino acids and a minimum length of 65 amino acids, provided that said peptidic sequence is liable to inhibit DNA replication, and/or to promote cellular differentiation, or
a peptidic sequence presenting a sequence identity of at least 30% with one of the above defined peptidic sequences, provided said peptidic sequence is liable to inhibit DNA replication and/or to provide cellular differentiation, said peptidic sequences being optionally in the form of a dimer,
in association with a pharmaceutically acceptable vehicle.

27. A pharmaceutical composition containing, as active substance, a nucleic acid coding for one of the peptidic sequences defined in claim 21, or its complementary sequence, or an antisense of the above-defined nucleic acid, in association with a pharmaceutically acceptable vehicle.

28. A pharmaceutical composition containing as active substance:
a nucleic acid sequence which comprises or is constituted of a nucleotide chain of at least 195 contiguous nucleotides selected from the nucleotide SEQ ID NO: 3, SEQ ID NO: 3 being delimited by the nucleotide in position 226 and by the nucleotide in position 480 of SEQ ID NO: 1, provided that, if present, the flanking regions of said nucleotide chain in said nucleic acid sequence are different from the flanking regions of said nucleotide chain in SEQ ID NO: 1, or
a nucleic acid sequence derived from the above-defined sequence by insertion, deletion or mutation, of at least one nucleotide in said nucleotide chain, provided that the resulting derived nucleic acid sequence has a maximum length of 255 nucleotides and a minimum length of 195 nucleotides, and provided that said derived nucleic acid codes for a peptidic sequence liable to inhibit DNA replication and/or to promote cellular differentiation, the resulting derived nucleic acid sequence being in particular such that it codes for a peptidic sequence in which the amino acids corresponding or homologous to the amino acids in positions 106, 109, 110, 112, 113, 114, 116, 118, 121, 123, 124, 125, and 128, of SEQ ID NO: 2 are not mutated, or
a nucleic acid presenting a sequence identity of at least 33% with one of the above defined nucleic acid sequences, provided that said nucleic acid sequence codes for a peptidic sequence liable to inhibit DNA replication and/or to promote cellular differentiation or its complementary sequence,
or the complementary sequence of one of the above-defined nucleic sequences
or an antisense of the above-defined sequences,
in association with a pharmaceutically acceptable vehicle.

29. A pharmaceutical composition, according to claim 28, containing, as active substance, a nucleic acid which comprises or is constituted of at least one of the following nucleotide chains:
SEQ ID NO: 3 delimited by the nucleotide in position 226 and the nucleotide in position 480 in SEQ ID NO: 1,
SEQ ID NO: 5 delimited by the nucleotide in position 244 and the nucleotide in position 480 in SEQ ID NO: 1,
SEQ ID NO: 7 delimited by the nucleotide in position 226 and the nucleotide in position 435 in SEQ ID NO: 1,
SEQ ID NO: 9 delimited by the nucleotide in position 229 and the nucleotide in position 435 in SEQ ID NO: 1,
SEQ ID NO: 11 delimited by the nucleotide in position 232 and the nucleotide in position 435 in SEQ ID NO: 1,
SEQ ID NO: 13 delimited by the nucleotide in position 235 and the nucleotide in position 435 in SEQ ID NO: 1,
SEQ ID NO: 15 delimited by the nucleotide in position 238 and the nucleotide in position 435 in SEQ ID NO: 1,
SEQ ID NO: 17 delimited by the nucleotide in position 241 and the nucleotide in position 435 in SEQ ID NO: 1,
provided that, if present, the flanking regions of said nucleotide chains in said nucleic acid are different from the flanking regions of said nucleotide chains in SEQ ID NO: 1, or
a nucleic acid sequence derived from the above-defined sequence by insertion, deletion or mutation of at least one nucleotide in said nucleotide chains, provided that the resulting derived nucleic acid sequence has a maximum length of 255 nucleotides and a minimum length of 195 nucleotides, and provided that said derived nucleic acid codes for a peptidic sequence liable to inhibit DNA replication and/or to promote cellular differentiation, the resulting derived peptidic sequence being in particular such that the amino acids corresponding or homologous to the amino acids in positions 106, 109, 110, 112, 113, 114, 116, 118, 121, 123, 124, 125, and 128, of SEQ ID NO: 2 are not mutated, or
a nucleic acid presenting a sequence identity of at least 33% with one of the above-defined sequences, provided that said nucleic acid sequence codes for a peptidic sequence liable to inhibit DNA replication and/or to promote cellular differentiation or its complementary sequence,
or the complementary sequence of one of the above-defined nucleic sequences,
or an antisense of the above-defined nucleic sequences,
in association with a pharmaceutically acceptable vehicle.

30. A peptide comprising or being constituted by one of the following peptidic chains:
SEQ ID NO: 4 delimited by amino acid in position 76 and amino acid in position 160 in SEQ ID NO: 2,
SEQ ID NO: 6 delimited by amino acid in position 82 and amino acid in position 160 in SEQ ID NO: 2,
SEQ ID NO: 8 delimited by amino acid in position 76 and amino acid in position 145 in SEQ ID NO: 2,
SEQ ID NO: 10 delimited by amino acid in position 77 and amino acid in position 145 in SEQ ID NO: 2,
SEQ ID NO: 12 delimited by amino acid in position 78 and amino acid in position 145 in SEQ ID NO: 2,
SEQ ID NO: 14 delimited by amino acid in position 79 and amino acid in position 145 in SEQ ID NO: 2,
SEQ ID NO: 16 delimited by amino acid in position 80 and amino acid in position 145 in SEQ ID NO: 2,
SEQ ID NO: 18 delimited by amino acid in position 81 and amino acid in position 145 in SEQ ID NO: 2,
provided that, if present, the flanking regions of said peptidic chains in said peptide are different from the flanking regions of said sequences in SEQ ID NO: 2, or
a peptidic sequence derived from the above-defined sequence by insertion, deletion or mutation, of at least one amino acid of said peptidic chains, provided that the resulting derived sequence has a maximum length of 85 amino acids and a minimum length of 65 amino acids, and provided that said peptidic sequence is liable to inhibit DNA replication, and/or to promote cellular differentiation, or
a peptidic sequence presenting a sequence identity of at least 30% with one of the above defined sequences, provided said peptidic sequence is liable to inhibit DNA replication and/or to provide cellular differentiation,

31. A nucleic acid coding for one of the peptidic sequences according to claim 30.

32. A nucleic acid hybridising to a nucleic acid sequence according to claim 31, or to its complementary sequence, under the following hybridisation conditions: 6×SSC, 0.5% SDS, 65° C.

33. A nucleic acid which comprises or is constituted of at least one of the following nucleotide chains:
SEQ ID NO: 3 delimited by the nucleotide in position 226 and the nucleotide in position 480 in SEQ ID NO: 1,
SEQ ID NO: 5 delimited by the nucleotide in position 244 and the nucleotide in position 480 in SEQ ID NO: 1,
SEQ ID NO: 7 delimited by the nucleotide in position 226 and the nucleotide in position 435 in SEQ ID NO: 1,
SEQ ID NO: 9 delimited by the nucleotide in position 229 and the nucleotide in position 435 in SEQ ID NO: 1,
SEQ ID NO: 11 delimited by the nucleotide in position 232 and the nucleotide in position 435 in SEQ ID NO: 1,
SEQ ID NO: 13 delimited by the nucleotide in position 235 and the nucleotide in position 435 in SEQ ID NO: 1,
SEQ ID NO: 15 delimited by the nucleotide in position 238 and the nucleotide in position 435 in SEQ ID NO: 1,
SEQ ID NO: 17 delimited by the nucleotide in position 241 and the nucleotide in position 435 in SEQ ID NO: 1,
provided that, if present, the flanking regions of said nucleotide chains in said nucleic acid are different from the flanking regions of said nucleotide chains in SEQ ID NO: 1, or
a nucleic acid sequence derived from the above-defined sequence by insertion, deletion or mutation of at least one nucleotide in said nucleotide chains, provided that the resulting derived nucleic acid sequence has a maximum length of 255 nucleotides and a minimum length of 195 nucleotides, and provided that said derived nucleic acid codes for a peptidic sequence liable to inhibit DNA replication and/or to promote cellular differentiation, or
a nucleic acid presenting a sequence identity of at least 33% with one of the above-defined sequences, provided that said nucleic acid sequence codes for a peptidic sequence liable to inhibit DNA replication and/or to promote cellular differentiation or its complementary sequence,
or the complementary sequence of one of the above-defined nucleic acid sequences,
or an antisense of the above-defined nucleic acid sequences.

34. A eukaryotic or prokaryotic expression vector comprising a nucleic acid such as defined according to claim 31, and the elements necessary for its expression in a eukaryotic or a prokaryotic cell.

35. A eukaryotic or prokaryotic cell transformed by a nucleic acid, or by a vector containing said nucleic acid, or by a vector containing said nucleic acid, wherein said nucleic acid is according to claim 31.

36. A polyclonal or monoclonal antibody, directed against a peptidic sequence according to claim 25.

37. An idiotypic antibody directed against the paratope of the antibody defined in claim 36.

38. A method for screening drugs liable to enhance DNA replication, in cells, comprising the following steps:
contacting a peptidic sequence according to claim 25 with a compound to screen,
selecting the compounds which bind to said peptidic sequence,
optionally checking that the selected compounds enhance DNA replication.

39. A method for screening drugs liable to enhance DNA replications comprising the following steps:
contacting a peptidic sequence according to claim 25 with a compound to screen and with a ligand of said peptidic sequence, such as an antibody, a scFv polypeptide, an aptamer, or the Cdt1 protein,
selecting the compounds which prevent the binding of the ligand to said peptidic sequence, and which do not bind to said ligand,
optionally checking that the selected compounds enhance DNA replication.

40. A method for screening drugs liable to inhibit DNA replication comprising the following steps:
contacting the Cdt1 protein and a peptidic sequence according to claim 25 with a compound to screen,
selecting the compounds which prevent binding of Cdt1 to said peptidic sequence, and which do not bind to said peptidic sequence,
optionally checking that the selecting compounds inhibit DNA replication.
